SEQUENCE LISTING (53 sequences)

SEQ ID NO: 1 - protein, 5 aa, Artificial
  anti-TA-MUC1 CDR H1
NYWMN

SEQ ID NO: 2 - protein, 5 aa, Artificial
  anti-TA-MUC1 CDR H1
DAWMD

SEQ ID NO: 3 - protein, 19 aa, Artificial
  anti-TA-MUC1 CDR H2
EIRLKSNNYTTHYAESVKG

SEQ ID NO: 4 - protein, 19 aa, Artificial
  anti-TA-MUC1 CDR H2
EIRSKANNHATYYAESVKG

SEQ ID NO: 5 - protein, 6 aa, Artificial
  anti-TA-MUC1 CDR H3
HYYFDY

SEQ ID NO: 6 - protein, 7 aa, Artificial
  anti-TA-MUC1 CDR H3
GGYGFDY

SEQ ID NO: 7 - protein, 118 aa, Artificial Sequence
  anti-TA-MUC1 heavy chain variable region
EVKLVESGGGLVQPGGSMKLSCAASGFTFSDAWMDWVRQSPEKGLEWVAELRSKANNHATYYAESVKGRFTLSRDVSKSSVYLQMNNLRAEDTGLYYCTRGGYGFDYWGQGTTLTVSS

SEQ ID NO: 8 - protein, 117 aa, Artificial Sequence
  anti-TA-MUC1 heavy chain variable region
EVKLVESGGGLVQPGGSMKLSCVASGFTFSNYWMNWVRQSPEKGLEWVAELRLKSNNYTTHYAESVKGRFTISRDDSKSSVSLQMNNLRVEDTGLYYCTRHYYFDYWGQGTTLTVSS

SEQ ID NO: 9 - protein, 117 aa, Artificial
  anti-TA-MUC1 heavy chain variable region
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 10 - protein, 16 aa, Artificial
  anti-TA-MUC1 CDR L1
RSSKSLLHSNGITYFF

SEQ ID NO: 11 - protein, 16 aa, Artificial
  anti-TA-MUC1 CDR L1
RSSQSIVHSNGNTYLE

SEQ ID NO: 12 - protein, 7 aa, Artificial
  anti-TA-MUC1 CDR L2
QMSNLAS

SEQ ID NO: 13 - protein, 7 aa, Artificial
  anti-TA-MUC1 CDR L2
KVSNRFS

SEQ ID NO: 14 - protein, 9 aa, Artificial
  anti-TA-MUC1 CDR L3
AQNLELPPT

SEQ ID NO: 15 - protein, 9 aa, Artificial
  anti-TA-MUC1 CDR L3
FQGSHVPLT

SEQ ID NO: 16 - protein, 114 aa, Artificial Sequence
  anti-TA-MUC1 light chain variable region
DLVLTQTPLSLPVSLGDQASLSCRSSQSLVHSNGNTYLEWYLQKPGQSPKLLLYKVSNRFSGVPDRFSGSGSGTDFTLKLSRVEAEDLGVYYCFQGSHVPLTFGDGTKLELKRA

SEQ ID NO: 17 - protein, 114 aa, Artificial Sequence
  anti-TA-MUC1 light chain variable region
DLVMTQAAFSNPVTLGTSASISCRSSKSLLHSNGLTYFFWYLQKPGLSPQLLLYQMSNLASGVPDRFSSSGSGTDFTLRLSRVEAEDVGVYYCAQNLELPPTFGGGTKLELKRA

SEQ ID NO: 18 - protein, 113 aa, Artificial
  anti-TA-MUC1 light chain vatiable region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKR

SEQ ID NO: 19 - protein, 4 aa, Artificial
  epitope
PDTR

SEQ ID NO: 20 - protein, 5 aa, Artificial
  epitope
PDTRP

SEQ ID NO: 21 - protein, 5 aa, Artificial Sequence
  anti-TFalpha CDR-H1
NYWLG

SEQ ID NO: 22 - protein, 17 aa, Artificial Sequence
  anti-TFalpha CDR-H2
DIYPGGGYTNYNEKFKG

SEQ ID NO: 23 - protein, 17 aa, Artificial Sequence
  anti-TFalpha CDR-H2
DIYPGGSYTNYNEKFKG

SEQ ID NO: 24 - protein, 10 aa, Artificial Sequence
  anti-TFalpha CDR-H3
YDAAGPWFAY

SEQ ID NO: 25 - protein, 10 aa, Artificial Sequence
  anti-TFalpha CDR-H3
YDAAGPGFAY

SEQ ID NO: 26 - protein, 8 aa, Artificial Sequence
  anti-TFalpha CDR-H3
YDNHYFDY

SEQ ID NO: 27 - protein, 119 aa, Artificial Sequence
  anti-TFalpha heavy chain variable region
QVQLKESGAELVRPGTSVKISCKASGYTFTNYWLGWVKQRPGHGLEWIGDIYPGGGYTNYNEKFKGKATLTADTSSSTAYMQLSSLTSEDSAVYFCAYYDAAGPGFAYWGQGTTVTVSS

SEQ ID NO: 28 - protein, 119 aa, Artificial Sequence
  anti-TFalpha heavy chain variable region
QVQLVQSGAEVKKPGASVKVSCKASGYTFTNYWLGWVRQAPGQGLEWIGDIYPGGGYTNYNEKFKGKATLTADTSTSTAYMELSSLRSEDSAVYFCAYYDAAGPWFAYWGQGTLVTVSS

SEQ ID NO: 29 - protein, 119 aa, Artificial Sequence
  anti-TFalpha heavy chain variable region
QVQLVQSGAEVKKPGASVKVSCKASGYTFTNYWLGWVRQAPGQGLEWIGDIYPGGGYTNYNEKFKGKATLTADTSSSTAYMQLSSLTSEDSAVYFCAYYDAAGPWFAYWGQGTLVTVSS

SEQ ID NO: 30 - protein, 119 aa, Artificial Sequence
  anti-TFalpha heavy chain variable region
QVQLVQSGAEVKKPGASVKVSCKASGYTFTNYWLGWVKQRPGQGLEWIGDIYPGGGYTNYNEKFKGKATLTADTSSSTAYMQLSSLTSEDSAVYFCAYYDAAGPWFAYWGQGTLVTVSS

SEQ ID NO: 31 - protein, 119 aa, Artificial Sequence
  anti-TFalpha heavy chain variable region
QVQLVQSGAEVKKPGASVKVSCKASGYTFTNYWLGWVKQRPGQGLEWIGDIYPGGGYTNYNEKFKGRVTITRDTSSSTAYMQLSSLTSEDSAVYFCAYYDAAGPWFAYWGQGTLVTVSS

SEQ ID NO: 32 - protein, 119 aa, Artificial Sequence
  anti-TFalpha heavy chain variable region
QVQLVQSGAEVKKPGASVKVSCKASGYTFTNYWLGWVRQAPGQGLEWIGDIYPGGGYTNYNEKFKGKATLTADTSTSTAYMELSSLRSEDTAVYYCARYDAAGPWFAYWGQGTLVTVSS

SEQ ID NO: 33 - protein, 16 aa, Artificial Sequence
  anti-TFalpha CDR-L1
RSSQSIVHSNGNTYLE

SEQ ID NO: 34 - protein, 16 aa, Artificial Sequence
  anti-TFalpha CDR-L1
RSSQSLLHSNGNTYLH

SEQ ID NO: 35 - protein, 16 aa, Artificial Sequence
  anti-TFalpha CDR-L1
KSSQSLLHSDGKTYLY

SEQ ID NO: 36 - protein, 7 aa, Artificial Sequence
  anti-TFalpha CDR-L2
KVSNRFS

SEQ ID NO: 37 - protein, 7 aa, Artificial Sequence
  anti-TFalpha CDR-L2
EVSSRFS

SEQ ID NO: 38 - protein, 9 aa, Artificial Sequence
  anti-TFalpha CDR-L3
FQGSHVPYT

SEQ ID NO: 39 - protein, 9 aa, Artificial Sequence
  anti-TFalpha CDR-L3
SQSTHVPYT

SEQ ID NO: 40 - protein, 113 aa, Artificial Sequence
  anti-TFalpha light chain variable region
DIQMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 41 - protein, 113 aa, Artificial Sequence
  anti-TFalpha light chain variable region
DIVMTQSPLSLPVTPGEPASISCRSSQSIVHSNGNTYLEWYLQKPGQSPKLLIYKVSNRFSGVPDRFSGSGSGTDFTLKISRVEAEDVGVYYCFQGSHVPYTFGQGTKVEIKR

SEQ ID NO: 42 - protein, 113 aa, Artificial Sequence
  anti-TFalpha light chain variable region
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPYTFGQGTKVEIKR

SEQ ID NO: 43 - protein, 6 aa, Artificial Sequence
  anti-CD3 CDR-H1
GYTFTR

SEQ ID NO: 44 - protein, 9 aa, Artificial Sequence
  anti-CD3 CDR-H2
YINPSRGYT

SEQ ID NO: 45 - protein, 10 aa, Artificial Sequence
  anti-CD3 CDR-H3
YYDDHYALDY

SEQ ID NO: 46 - protein, 119 aa, Artificial Sequence
  anti-CD3 heavy chain variable region
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYALDYWGQGTTLTVSS

SEQ ID NO: 47 - protein, 10 aa, Artificial Sequence
  anti-CD3 CDR-L1
RASSSVSYMN

SEQ ID NO: 48 - protein, 7 aa, Artificial Sequence
  anti-CD3 CDR-L2
DTSKVAS

SEQ ID NO: 49 - protein, 9 aa, Artificial Sequence
  anti-CD3 CDR-L3
QQWSSNPLT

SEQ ID NO: 50 - protein, 106 aa, Artificial Sequence
  anti-CD3 light chain variable region
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 51 - protein, 5 aa, Artificial Sequence
  linker repeat
GGGGS

SEQ ID NO: 52 - protein, 113 aa, Artificial Sequence
  anti-TA-MUC1 light chain variable region
DLVLTQTPLSLPVSLGDQASLSCRSSQSLVHSNGNTYLEWYLQKPGQSPKLLLYKVSNRFSGVPDRFSGSGSGTDFTLKLSRVEAEDLGVYYCFQGSHVPLTFGDGTKLELKR

SEQ ID NO: 53 - protein, 113 aa, Artificial Sequence
  anti-TA-MUC1 light chain variable region
DLVMTQAAFSNPVTLGTSASISCRSSKSLLHSNGLTYFFWYLQKPGLSPQLLLYQMSNLASGVPDRFSSSGSGTDFTLRLSRVEAEDVGVYYCAQNLELPPTFGGGTKLELKR

(19) United States
(12) Patent Application Publication
(10) Pub. No.: US 2020/0131275 A1
(43) Pub. Date: Apr. 30, 2020

(54) MULTISPECIFIC ANTIBODY CONSTRUCTS BINDING TO MUC1 AND CD3

(71) Applicant: Glycotope GmbH, Berlin (DE)

(72) Inventors: Steffen GOLETZ, Berlin (DE); Patrik KEHLER, Berlin (DE); Johanna GELLERT, Berlin (DE); Antje DANIELCZYK, Berlin (DE); Anika JÄKEL, Berlin (DE)

(21) Appl. No.: 16/494,566

(22) PCT Filed: Mar. 27, 2018
(86) PCT No.: PCT/EP2018/057721

§ 371 Date: Sep. 16, 2019

(30) Foreign Application Priority Data
Mar. 29, 2017 (LU) LU100152

(51) Int. Cl. C07K 16/30; C07K 16/28; C07K 16/32; G01N 33/574

(52) U.S. Cl. CPC C07K 16/3092; C07K 16/2809; C07K 16/2896; C07K 16/32; G01N 33/574; C07K 2317/76; C07K 2317/41; C07K 2317/622; C07K 2317/72; C07K 2317/732; C07K 2317/31

(57) ABSTRACT

The present invention pertains to multispecific antibody constructs directed against the cancer antigen MUC1 and the T cells antigen CD3. In particular, the multispecific antibody constructs recruit T cells to the cancer site. The design of the multispecific antibody constructs show strong antigen binding and high T cell activation.

### FIELD OF THE INVENTION

The present invention pertains to the field of antibodies. A multispecific antibody construct directed against a cancer antigen and an immune cell antigen is provided. In particular, the multispecific antibody construct recruits T cells—by binding to CD3—to the cancer site—by binding to the cancer antigen MUC1. The design of the multispecific antibody construct shows distinct advantages in the specific setting provided, in particular strong antigen binding and high T cell activation. In specific embodiments, the present invention is directed to the therapeutic and diagnostic use of these multispecific antibody constructs.

### BACKGROUND OF THE INVENTION

Antibodies against tumor-associated antigens are widely used therapeutics against cancers. Today, many anti-cancer antibodies are approved for human therapy. Some of these antibodies act by blocking certain signaling pathways which are critical for survival or proliferation of specific cancer cells. Other anti-cancer antibodies activate the patient's immune response against the targeted cancer cells, for example by initiating antibody-dependent cellular cytotoxicity (ADCC) via natural killer cells. This mechanism is induced by binding of the antibody's Fc part to Fc receptors on the immune cells.

An interesting and important group of antibodies are those directed against mucin proteins. Mucins are a family of high molecular weight, heavily glycosylated proteins produced by many epithelial tissues in vertebrates. They can be subdivided into mucin proteins which are membrane-bound due to the presence of a hydrophobic membrane-spanning domain that favors retention in the plasma membrane, and mucins which are secreted onto mucosal surfaces or secreted to become a component of saliva. The human mucin protein family consists of many family members, including membrane bound MUC1.

Increased mucin production occurs in many adenocarcinomas, including cancer of the pancreas, lung, breast, ovary, colon, etc. Mucins are also overexpressed in lung diseases such as asthma, bronchitis, chronic obstructive pulmonary disease or cystic fibrosis. Two membrane mucins, MUC1 and MUC4 have been extensively studied in relation to their pathological implication in the disease process. Moreover, mucins are also being investigated for their potential as diagnostic markers. Several antibodies directed against mucin proteins, in particular MUC1, are known in the art. However, their therapeutic efficacy could still be improved.

In view of this, there is a need in the art to provide therapeutic anti-MUC1 antibody constructs with increased therapeutic efficacy.

### SUMMARY OF THE INVENTION

The present inventors have found that anti-MUC1 antibodies can be improved by combining them with anti-CD3 antigen binding fragments. CD3 is a characteristic surface protein of T cells, which together with the T cell receptor (TCR) forms the TCR-CD3 complex. Simultaneous binding of the anti-cancer antigen MUC1 and the T cell antigen CD3 enables recruitment of cytotoxic T lymphocytes (CTLs) to the tumor site. The recruitment of CTLs is of interest since they are among the most potent cells that mediate antitumor effects. Surprisingly, the present inventors found that a specific design of multispecific antibody constructs binding to MUC1 and to CD3 results in significantly stronger CD3 binding and also markedly increased T cells activation. In this superior design, an anti-CD3 antigen binding fragment is fused to the C terminus of the heavy chain of an anti-MUC1 antibody module. As demonstrated experimentally, this construct design binds stronger to CD3 and induces increased T cell-mediated ADCC and T cell proliferation compared to similar antibody constructs wherein the anti-CD3 antigen binding fragment is fused to the C terminus of the light chain of the anti-MUC1 antibody module.

Therefore, in a first aspect, the present invention is directed to a multispecific antibody construct, comprising
(i) an anti-MUC1 antibody module, the antibody module comprising at least one antibody heavy chain, and
(ii) an anti-CD3 antigen binding fragment;
wherein the antigen binding fragment is fused to the C terminus of the heavy chain of the antibody module.

In a second aspect, the present invention provides a nucleic acid encoding the multispecific antibody construct according to the invention. Furthermore, in a third aspect an expression cassette or vector comprising the nucleic acid according to the invention and a promoter operatively connected with said nucleic acid and, in a fourth aspect, a host cell comprising the nucleic acid or the expression cassette or vector according to the invention are provided.

In a fifth aspect, the present invention is directed to a pharmaceutical composition comprising the multispecific antibody construct according to the invention.

According to a sixth aspect, the invention provides the multispecific antibody construct or the pharmaceutical composition according to the invention for use in medicine, in particular in the treatment of cancer, infections, graft-versus-host disease or autoimmune diseases.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. In particular, the amino acid sequence of the constant domains of a γ-type (especially γ1-type) heavy chain is at least 95%, especially at least 98%, identical to the amino acid sequence of the constant domains of a human γ (especially one of the allotypes of the human γ1) antibody heavy chain. Furthermore, the amino acid sequence of the constant domain of a κ-type light chain is in particular at least 95%, especially at least 98%, identical to the amino acid sequence of the constant domain of one of the allotypes of the human K antibody light chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The antigen-binding portion of an antibody usually refers to full length or one or more fragments of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and CH1 domains; a Fv fragment consisting of the V_{L} and V_{H} domains of a single arm of an antibody; and a dAb fragment, which consists of a V_{H} domain.

The “Fab part” of an antibody in particular refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. Preferably, “Fab part” refers to that part of an antibody corresponding to the fragment obtained by digesting a natural antibody with papain which contains the antigen binding activity of the antibody.

In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions C_{H2}, C_{H3} and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

The terms “antibody” and “antibody construct”, as used herein, refer in certain embodiments to a population of antibodies or antibody constructs, respectively, of the same kind. In particular, all antibodies or antibody constructs of the population exhibit the features used for defining the antibody or antibody construct. In certain embodiments, all antibodies or antibody constructs in the population have the same amino acid sequence. Reference to a specific kind of antibody or antibody construct, such as a multispecific antibody construct specifically binding to an epitope of MUC1 and to an epitope of CD3, in particular refers to a population of this kind of antibody or antibody construct.

The term “antibody” as used herein also includes fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. Derivatives of an antibody in particular include antibodies which bind to the same antigen as the parent antibody, but which have a different amino acid sequence than the parent antibody from which it is derived. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, preferably at least 80%, at least 85%, at least 90%, at least 93%, at least 95%, at least 97%, at least 98% or at least 99% homologous or identical, especially identical, to the respective amino acid sequences of the parent antibody. In certain embodiments, the antibody derived from (i.e. derivative of) a parent antibody comprises the same CDR sequences as the parent antibody, but differs in the remaining sequences of the variable regions.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-MUC1 antibody” in particular refers to an antibody specifically binding MUC1 and preferably is capable of binding to MUC1 with an affinity having a Kₐ of at least 10⁶ M⁻¹, preferably at least 10⁷ M⁻¹, more preferably at least 10⁸ M⁻¹.

An “antibody module” as referred to herein refers to a polypeptide construct which is derived from an antibody and is capable of specifically binding to an antigen. In particular, the antibody module comprises at least one, especially two, antibody heavy chains and optionally at least one, especially two, antibody light chains.

The term “antigen binding fragment” as used herein refers to a polypeptide construct which is derived from an antibody, is capable of specifically binding to an antigen, but does not comprise all elements of a natural antibody. In particular, the antigen binding fragment does not comprise some or all of the constant domains of an antibody, and may comprise only one instead of two antigen binding sites. “CD3” refers to the T cell co-receptor CD3 (cluster of differentiation 3), in particular to human CD3. It is generally composed of four distinct polypeptide chains, a CD3γ chain, a CD3δ chain, and two CD3ε chains. Together with the T cell receptor (TCR), CD3 may form the TCR-CD3 complex.

The term “MUC1” refers to the protein MUC1, also known as mucin-1, polymorphic epithelial mucin (PEM) or cancer antigen 15-3, in particular to human MUC1. MUC1 is a member of the mucin family and encodes a membrane bound, glycosylated phosphoprotein. MUC1 has a core protein mass of 120-225 kDa which increases to 250-500 kDa with glycosylation. It extends 200-500 nm beyond the surface of the cell. The protein is anchored to the apical surface of many epithelial cells by a transmembrane domain. The extracellular domain includes a 20 amino acid variable number tandem repeat (VNTR) domain, with the number of repeats varying from 20 to 120 in different individuals. These repeats are rich in serine, threonine and proline residues which permits heavy O-glycosylation. In certain embodiments, the term “MUC1” refers to tumor-associated MUC1 (“TA-MUC1”). TA-MUC1 is MUC1 present on cancer cells. This MUC1 differs from MUC1 present on non-cancer cells in its much higher expression level, its localization and its glycosylation. In particular, TA-MUC1 is present apolarly over the whole cell surface in cancer cells, while in non-cancer cells MUC1 has a strictly apical expression and hence, is not accessible for systemically administered antibodies. Furthermore, TA-MUC1 has an aberrant O-glycosylation which exposes new peptide epitopes on the MUC1 protein backbone and new carbohydrate tumor antigens such as the Thomsen-Friedenreich antigen alpha (TFα).

“TFα”, also called Thomsen-Friedenreich antigen alpha or Core-1, refers to the disaccharide Gal-ß1,3-GalNAc which is O-glycosidically linked in an alpha-anomeric configuration to the hydroxy amino acids serine or threonine of proteins in carcinoma cells.

The term “sialic acid” in particular refers to any N- or O-substituted derivatives of neuraminic acid. It may refer to both 5-N-acetylneuraminic acid and 5-N-glycolylneuraminic acid, but preferably only refers to 5-N-acetylneuraminic acid. The sialic acid, in particular the 5-N-acetylneuraminic acid preferably is attached to a carbohydrate chain via a 2,3- or 2,6-linkage. Preferably, in the antibodies described herein both 2,3- as well as 2,6-coupled sialic acids are present.

A “relative amount of glycans” according to the invention refers to a specific percentage or percentage range of the glycans attached to the antibodies of an antibody preparation or in a composition comprising antibodies, respectively. In particular, the relative amount of glycans refers to a specific percentage or percentage range of all glycans comprised in the antibodies and thus, attached to the polypeptide chains of the antibodies in an antibody preparation or in a composition comprising antibodies. 100% of the glycans refers to all glycans attached to the antibodies of the antibody preparation or in a composition comprising antibodies, respectively. For example, a relative amount of glycans carrying bisecting GlcNAc of 10% refers to a composition comprising antibodies wherein 10% of all glycans comprised in the antibodies and thus, attached to the antibody polypeptide chains in said composition comprise a bisecting GlcNAc residue while 90% of all glycans comprised in the antibodies and thus, attached to the antibody polypeptide chains in said composition do not comprise a bisecting GlcNAc residue. The corresponding reference amount of glycans representing 100% may either be all glycan structures attached to the antibodies in the composition, or all N-glycans, i.e. all glycan structures attached to an asparagine residue of the antibodies in the composition, or all complex-type glycans. The reference group of glycan structures generally is explicitly indicated or directly derivable from the circumstances by the skilled person.

The term “N-glycosylation” refers to all glycans attached to asparagine residues of the polypeptide chain of a protein. These asparagine residues generally are part of N-glycosylation sites having the amino acid sequence Asn-Xaa-Ser/Thr, wherein Xaa may be any amino acid except for proline. Likewise, “N-glycans” are glycans attached to asparagine residues of a polypeptide chain. The terms “glycan”, “glycan structure”, “carbohydrate”, “carbohydrate chain” and “carbohydrate structure” are generally used synonymously herein. N-glycans generally have a common core structure consisting of two N-acetylglucosamine (GlcNAc) residues and three mannose residues, having the structure Manα1,6-(Manα1,3-)Manβ1,4-GlcNAcβ1,4-GlcNAcβ1-Asn with Asn being the asparagine residue of the polypeptide chain. N-glycans are subdivided into three different types, namely complex-type glycans, hybrid-type glycans and high mannose-type glycans.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA.

According to the invention, the term “host cell” relates to any cell which can be transformed or transfected with an exogenous nucleic acid. The term “host cells” comprises according to the invention prokaryotic (e.g. E. coli) or eukaryotic cells (e.g. mammalian cells, in particular human cells, yeast cells and insect cells). Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

The term “cancer” according to the invention in particular comprises leukemias, seminomas, melanomas, carcinomas, teratomas, lymphomas, sarcomas, mesotheliomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, bladder cancer, cancer of the uterus, ovarian cancer and lung cancer and the metastases thereof. The term cancer according to the invention also comprises cancer metastases.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of bispecific antibody constructs in which scFv fragments specifically binding CD3 were fused to anti-cancer antibodies targeting MUC1. The established anti-MUC1 antibodies exert their anti-cancer activity by binding to tumor-associated MUC1 and recruiting and activating cytotoxic immune cells. The binding and activation of immune cells, in particular natural killer cells (NK cells) is achieved via the interaction of the antibody Fc part with Fcγ receptors, especially FcγRIIIa, on the immune cells. Upon activation, antibody-dependent cellular cytotoxicity (ADCC) is initiated. Killing of tumor cells may also be mediated by the highly efficient cytotoxic T lymphocytes. In view of this, the present inventors further improved efficacy of the established anti-MUC1 antibodies by attaching anti-CD3 binding fragments to these antibodies. CD3 is a cell surface protein of cytotoxic T lymphocytes and the anti-CD3 binding fragments recruit said cytotoxic T lymphocytes to the tumor site targeted by the anti-MUC1 antibodies.

The present inventors surprisingly found in this respect that the attachment of the anti-CD3 binding fragment to the C terminus of the heavy chain of the anti-MUC1 antibodies results in a significantly improved CD3 binding and T cell activation, including T cell mediated ADCC activity and T cell proliferation. Bispecific constructs comprising the anti-CD3 binding fragment at the C terminus of the light chain of the anti-MUC1 antibodies, in contrast, showed reduced CD3 binding and T cell activation. This was highly surprising since in the relevant technical field short distances between the T cell binding domain and the cancer cell binding domain were strongly favored (see, e.g., Bluemel, C. et al. (2010) Cancer Immunol Immunother 59:1197-1209; Chames, P. and Baty, D. (2009) mAbs 1(6):539-547). In contrast to this, the distance between the binding domains is much larger in the antibody constructs of the present invention wherein the anti-CD3 binding fragment is attached to the C terminus of the heavy chain, compared to constructs wherein the anti-CD3 binding fragment is attached to the C terminus of the light chain. In a control construct targeting the tumor antigen HER2 and CD3, indeed the short distance between the two binding sites was favorable as taught in the art. Here, T cell activation was stronger for constructs bearing the CD3 binding site at the light chain C terminus compared to at the heavy chain C terminus.

This teaching was verified with two different anti-MUC1 antibodies. PankoMab specifically binds to the TA-MUC1 epitope in the extracellular repeat region of MUC1, which is only accessible on tumor cells. KaroMab binds to the Thomson-Friedenreich antigen, a carbohydrate structure present on MUC1 which is also only accessible on tumor cells. For the anti-cancer antibodies PankoMab and KaroMab, superior CD3 binding and T cell activation were obtained with antibody constructs wherein the anti-CD3 scFv fragments were fused to the C terminus of the heavy chains of the anti-cancer antibodies.

In view of these findings, the present invention provides a multispecific antibody construct, comprising
(i) an anti-MUC1 antibody module, the antibody module comprising at least one antibody heavy chain, and
(ii) an anti-CD3 antigen binding fragment;
   wherein the antigen binding fragment is fused to the C terminus of the heavy chain of the antibody module.

The multispecific antibody construct is a protein construct which comprises two or more different antigen binding sites specifically binding to different antigens. At least one antigen binding site specifically binds to an epitope of MUC1 and at least one other antigen binding site specifically binds to CD3. The multispecific antibody construct may comprise more than one antigen binding site specifically binding to an epitope of MUC1, which antigen binding sites may have the same amino acid sequence. In particular, all antigen binding sites specifically binding to an epitope of MUC1 are part of the anti-MUC1 antibody module. Furthermore, the multispecific antibody construct may comprise more than one antigen binding site specifically binding to CD3, which antigen binding sites may have the same amino acid sequence. The antigen binding sites specifically binding to CD3 may be part of the same antigen binding fragment or may be present in separate antigen binding fragments.

An antigen binding site comprises at least one antibody variable region, in particular an antibody heavy chain variable region. In specific embodiments, an antigen binding site comprises an antibody heavy chain variable region and an antibody light chain variable region. The antibody heavy chain variable region and the antibody light chain variable region of an antigen binding site may be fused to each other via a peptide linker. In certain embodiments, an antigen binding site is a single chain variable region fragment (scFv).

The Anti-MUC1 Antibody Module

The anti-MUC1 antibody module comprises at least one antigen binding site specifically binding to an epitope of MUC1. In certain embodiments, the antibody module comprises at least two, especially exactly two, antigen binding sites specifically binding to an epitope of MUC1. These antigen binding sites may be different or identical and in particular have the same amino acid sequence. In specific embodiments, the antigen binding sites of the antibody module comprise an antibody heavy chain variable region and an antibody light chain variable region.

The anti-MUC1 antibody module comprises at least one antibody heavy chain. In certain embodiments, the antibody module comprises two antibody heavy chains. The antibody heavy chains in particular comprise a VH domain, a CH1 domain, a hinge region, a CH2 domain and a CH3 domain. In certain other embodiments, the antibody heavy chains comprise a CH2 domain and a CH3 domain, but do not comprise a CH1 domain. In further embodiments, one or more constant domains of the heavy chains may be replaced by other domains, in particular similar domains such as for example albumin. The antibody heavy chains may be of any type, including γ-, α-, ε-, δ- and μ-chains, and preferably are γ-chains, including γ1-, γ2-, γ3- and γ4-chains, especially γ1-chains. Hence, the antibody module preferably is an IgG-type antibody module, in particular an IgG1-type antibody module.

In preferred embodiments, the antibody module comprises an Fc region. The antibody module may especially be a whole antibody, comprising two heavy chains each comprising the domains VH, CH1, hinge region, CH2 and CH3, and two light chains each comprising the domains VL and CL. The antibody module in particular is capable of binding to one or more human Fcγ receptors, especially human Fcγ receptor IIIA. In certain embodiments, the antibody module does not or not significantly bind the human Fcγ receptor IIIA. In these embodiments the antibody module in particular does not comprise a glycosylation site in the CH2 domain.

In particular, the antibody module further comprises at least one antibody light chain, especially two antibody light chains. The antibody light chains in particular comprise a VL domain and a CL domain. The antibody light chain may be a κ-chain or a λ-chain and especially is a κ-chain. In certain embodiments, the antibody module comprises two antibody heavy chains and two antibody light chains.

In alternative embodiments, the antibody module does not comprise an antibody light chain. In these embodiments, the antibody heavy chains of the antibody module may additionally comprise a light chain variable region. In particular, the light chain variable region is fused to the N terminus of the heavy chain or is inserted C terminal to the heavy chain variable region. Peptide linkers may be present to connect the light chain variable region with the remaining parts of the heavy chain.

The anti-MUC1 antibody module specifically binds to an epitope of MUC1. The epitope may be specific for MUC1, i.e. it is not present on other molecules, or it may be an epitope also found on other molecules.

In certain embodiments, the antibody module binds to MUC1 in a glycosylation-dependent manner. In particular, the antibody module binds stronger to MUC1 if it is glycosylated, especially glycosylated in the extracellular tandem repeats. In specific embodiments, the antibody module binds stronger to MUC1 if it is O-glycosylated with N-acetyl galactosamine (Tn), sialyl α2-6 N-acetyl galactosamine (sTn), galactose ß1-3 N-acetyl galactosamine (TF) or galactose ß1-3 (sialyl α2-6)N-acetyl galactosamine (sTF), preferably with Tn or TF.

In certain embodiments, the antibody module specifically binds to an epitope in the extracellular tandem repeats of MUC1. In particular, the antibody module binds stronger if said tandem repeats are glycosylated at a threonine residue with N-acetyl galactosamine (Tn), sialyl α2-6 N-acetyl galactosamine (sTn), galactose β1-3 N-acetyl galactosamine (TF) or galactose β1-3 (sialyl α2-6)N-acetyl galactosamine (sTF), preferably with Tn or TF. Preferably, the carbohydrate moiety is bound to the threonine residue by an α-O-glycosidic bond.

In particular embodiments, the antibody module is capable of specifically binding to an epitope in the tandem repeat domain of MUC1 which comprises the amino acid sequence PDTR (SEQ ID NO: 19) or PDTRP (SEQ ID NO: 20). The binding to this epitope preferably is glycosylation dependent, as described above, wherein in particular the binding is increased if the carbohydrate moiety described above is attached to the threonine residue of the sequence PDTR or PDTRP (SEQ ID NOs: 19 and 20), respectively.

In certain embodiments, the antibody module specifically binds a tumor-associated MUC1 epitope (TA-MUC1). A TA-MUC1 epitope in particular refers to an epitope of MUC1 which is present on tumor cells but not on normal cells and/or which is only accessible by antibodies in the host's circulation when present on tumor cells but not when present on normal cells. The epitopes described above, in particular those present in the tandem repeat domain of MUC1, may be tumor-associated MUC1 epitopes. In certain embodiments, the binding of the antibody module to cells expressing TA-MUC1 epitope is stronger than the binding to cells expressing normal, non-tumor MUC1. Preferably, said binding is at least 1.5-fold stronger, preferably at least 2-fold stronger, at least 5-fold stronger, at least 10-fold stronger or at least 100-fold stronger. In particular, TA-MUC1 is glycosylated with at least one N-acetyl galactosamine (Tn) or galactose ß1-3 N-acetyl galactosamine (TF) in its extracellular tandem repeat region. In certain embodiments, the antibody module specifically binds to this epitope in the extracellular tandem repeat region of TA-MUC1 comprising N-acetyl galactosamine (Tn) or galactose ß1-3 N-acetyl galactosamine (TF). Especially, said epitope comprises at least one PDTR or PDTRP (SEQ ID NO: 19 or 20) sequence of the MUC1 tandem repeats and is glycosylated at the threonine of the PDTR or PDTRP (SEQ ID NO: 19 or 20) sequence with N-acetyl galactosamine (Tn) or galactose ß1-3 N-acetyl galactosamine (TF), preferably via an α-O-glycosidic bond. For TA-MUC1 binding, the antibody module preferably specifically binds the glycosylated MUC1 tumor epitope such that the strength of the bond is increased at least by a factor 2, preferably a factor of 4 or a factor of 10, most preferably a factor of 20 in comparison with the bond to the non-glycosylated peptide of identical length and identical peptide sequence.

In the following, specific embodiments of antibody modules specifically binding to TA-MUC1 are described.

In certain embodiments, the antibody module comprises at least one heavy chain variable region comprising the complementarity determining regions CDR-H1 having the amino acid sequence of SEQ ID NO: 1, CDR-H2 having the amino acid sequence of SEQ ID NO: 3 and CDR-H3 having the amino acid sequence of SEQ ID NO: 5, or comprising the complementarity determining regions CDR-H1 having the amino acid sequence of SEQ ID NO: 2, CDR-H2 having the amino acid sequence of SEQ ID NO: 4 and CDR-H3 having the amino acid sequence of SEQ ID NO: 6. According to one embodiment, the heavy chain variable region(s) present in the antibody module comprise(s) the amino acid sequence of SEQ ID NOs: 7, 8 or 9 or an amino acid sequence which is at least 75%, in particular at least 80%, at least 85%, at least 90%, at least 95% or at least 97% identical to one of said sequences. In certain embodiments, the heavy chain variable region of the antibody module comprises an amino acid sequence (i) which comprises a set of CDRs wherein CDR-H1 has the amino acid sequence of SEQ ID NO: 1, CDR-H2 has the amino acid sequence of SEQ ID NO: 3 and CDR-H3 has the amino acid sequence of SEQ ID NO: 5, or wherein CDR-H1 has the amino acid sequence of SEQ ID NO: 2, CDR-H2 has the amino acid sequence of SEQ ID NO: 4 and CDR-H3 has the amino acid sequence of SEQ ID NO: 6; and (ii) which is at least 80%, at least 85%, at least 90%, or at least 95% identical to any one of SEQ ID NOs: 7, 8 and 9, especially 9.

The antibody module may further comprise at least one light chain variable region comprising the complementarity determining regions CDR-L1 having the amino acid sequence of SEQ ID NO: 10, CDR-L2 having the amino acid sequence of SEQ ID NO: 12 and CDR-L3 having the amino acid sequence of SEQ ID NO: 14, or comprising the complementarity determining regions CDR-L1 having the amino acid sequence of SEQ ID NO: 11, CDR-L2 having the amino acid sequence of SEQ ID NO: 13 and CDR-L3 having the amino acid sequence of SEQ ID NO: 15. According to one embodiment, the light chain variable region(s) present in the antibody module comprise(s) the amino acid sequence of SEQ ID NOs: 16, 17, 18, 52 or 53 or an amino acid sequence which is at least 75%, in particular at least 80%, at least 85%, at least 90%, at least 95% or at least 97% identical to one of said sequences. In certain embodiments, the light chain variable region of the antibody module comprises an amino acid sequence (i) which comprises a set of CDRs wherein CDR-L1 has the amino acid sequence of SEQ ID NO: 10, CDR-L2 has the amino acid sequence of SEQ ID NO: 12 and CDR-L3 has the amino acid sequence of SEQ ID NO: 14, or wherein CDR-L1 has the amino acid sequence of SEQ ID NO: 11, CDR-L2 has the amino acid sequence of SEQ ID NO: 13 and CDR-L3 has the amino acid sequence of SEQ ID NO: 15; and (ii) which is at least 80%, at least 85%, at least 90%, or at least 95% identical to any one of SEQ ID NOs: 16, 17, 18, 52 and 53, especially 18.

In particular preferred embodiments, the antibody module comprises at least one, in particular two, heavy chain variable region comprising the amino acid sequence of SEQ ID NO: 9 and at least one, in particular two, light chain variable region comprising the amino acid sequence of SEQ ID NO: 18. In a further embodiment, the antibody module is derived from an antibody comprising one or more of the sequences described above, in particular from the antibody PankoMab in its chimeric or humanized version as described, for example, in WO 2004/065423 and WO 2011/012309, or from the antibody Gatipotuzumab.

In specific embodiments, the antibody module specifically binds to the Thomsen-Friedenreich antigen TFα. TFα is the disaccharide structure Gal-ß1,3-GalNAc which is O-glycosidically linked in an α-anomeric configuration to the hydroxy amino acids serine or threonine of proteins in carcinoma cells. One of the main carriers of TFα in cancer cells is MUC1. TFα especially is present at the O-glycosylation sites of the tandem repeats in the extracellular region of MUC1. Hence, the antibody module especially binds to TFα on MUC1.

In the following, specific embodiments of antibody modules specifically binding to TFα are described.

In certain embodiments, the antibody module comprises at least one heavy chain variable region comprising the complementarity determining regions CDR-H1 having the amino acid sequence of SEQ ID NO: 21, CDR-H2 having the amino acid sequence of SEQ ID NO: 22 or 23 and CDR-H3 having the amino acid sequence of SEQ ID NO: 24, 25 or 26. In particular, the heavy chain variable region comprises the CDRs having the amino acid sequences of SEQ ID NOs: 21, 22 and 24. According to one embodiment, the heavy chain variable region(s) present in the antibody module comprise(s) the amino acid sequence of any one of SEQ ID NOs: 27 to 32 or an amino acid sequence which is at least 75%, in particular at least 80%, at least 85%, at least 90%, at least 95% or at least 97% identical to one of said sequences. In certain embodiments, the heavy chain variable region of the antibody module comprises an amino acid sequence (i) which comprises a set of CDRs wherein CDR-H1 has the amino acid sequence of SEQ ID NO: 21, CDR-H2 has the amino acid sequence of SEQ ID NO: 22 and CDR-H3 has the amino acid sequence of SEQ ID NO: 24 or 25; and (ii) which is at least 80%, at least 85%, at least 90%, or at least 95% identical to any one of SEQ ID NOs: 27 to 32.

The antibody module may further comprise at least one light chain variable region comprising the complementarity determining regions CDR-L1 having the amino acid sequence of SEQ ID NO: 33, 34 or 35, CDR-L2 having the amino acid sequence of SEQ ID NO: 36 or 37 and CDR-L3 having the amino acid sequence of SEQ ID NO: 38 or 39. In particular, the heavy chain variable region comprises the CDRs having the amino acid sequences of SEQ ID NOs: 33, 36 and 38. According to one embodiment, the light chain variable region(s) present in the antibody module comprise(s) the amino acid sequence of any one of SEQ ID NOs: 40, 41 and 42 or an amino acid sequence which is at least 75%, in particular at least 80%, at least 85%, at least 90%, at least 95% or at least 97% identical to one of said sequences. In certain embodiments, the light chain variable region of the antibody module comprises an amino acid sequence (i) which comprises a set of CDRs wherein CDR-L1 has the amino acid sequence of SEQ ID NO: 33, CDR-L2 has the amino acid sequence of SEQ ID NO: 36 and CDR-L3 has the amino acid sequence of SEQ ID NO: 38; and (ii) which is at least 80%, at least 85%, at least 90%, or at least 95% identical to any one of SEQ ID NOs: 40, 41 and 42.

In particular preferred embodiments, the antibody module comprises at least one, in particular two, heavy chain variable region comprising the amino acid sequence of SEQ ID NO: 27, 29, 30 or 31 and at least one, in particular two, light chain variable region comprising the amino acid sequence of SEQ ID NO: 40, 41 or 42. In a further embodiment, the antibody module is derived from an antibody comprising one or more of the sequences described above.

The Anti-CD3 Antigen Binding Fragment

The anti-CD3 antigen binding fragment comprises at least one antigen binding site specific for CD3. In particular, the antigen binding fragment comprises an antibody heavy chain variable region (VH domain). In specific embodiments, the antigen binding fragment additionally comprises an antibody light chain variable region (VL domain) which together with the VH domain forms an antigen binding site. The VH domain and the VL domain may be formed by the same polypeptide chain or by separate polypeptide chains. In particular, they are formed by the same polypeptide chain and in particular the antigen binding fragment is a single chain fragment. The VH domain and the VL domain may be linked to each other via a peptide linker. A peptide linker as described herein may be used to link the VH domain and the VL domain to each other. In the antigen binding fragment comprising a VH domain and a VL domain formed by a single polypeptide chain, the VH domain may be N terminal or C terminal of the VL domain, and in particular is N terminal of the VL domain. In certain embodiments, the antigen binding fragment is a single chain variable region fragment (scFv) specifically binding to CD3.

The anti-CD3 antigen binding fragment may comprise more than one antigen binding site, but in particular comprises only one antigen binding site. In certain embodiments, the antigen binding fragment does not comprise any antibody constant region domains.

The antigen binding fragment specifically binds to an epitope of CD3. In particular, the antigen binding fragment specifically binds to CD3ε. In specific embodiments, the antigen binding fragment specifically binds to CD3ε in a conformation-dependent manner, especially only if it is in complex with CD3δ.

In certain embodiments, the antigen binding fragment comprises at least one heavy chain variable region comprising the complementarity determining regions CDR-H1 having the amino acid sequence of SEQ ID NO: 43, CDR-H2 having the amino acid sequence of SEQ ID NO: 44 and CDR-H3 having the amino acid sequence of SEQ ID NO: 45. According to one embodiment, the heavy chain variable region(s) present in the antigen binding fragment comprise(s) the amino acid sequence of SEQ ID NOs: 46 or an amino acid sequence which is at least 75%, in particular at least 80%, at least 85%, at least 90%, at least 95% or at least 97% identical to one of said sequences. In certain embodiments, the heavy chain variable region of the antigen binding fragment comprises an amino acid sequence (i) which comprises a set of CDRs wherein CDR-H1 has the amino acid sequence of SEQ ID NO: 43, CDR-H2 has the amino acid sequence of SEQ ID NO: 44 and CDR-H3 has the amino acid sequence of SEQ ID NO: 45; and (ii) which is at least 80%, at least 85%, at least 90%, or at least 95% identical to any one of SEQ ID NOs: 46.

The antigen binding fragment may further comprise at least one light chain variable region comprising the complementarity determining regions CDR-L1 having the amino acid sequence of SEQ ID NO: 47, CDR-L2 having the amino acid sequence of SEQ ID NO: 48 and CDR-L3 having the amino acid sequence of SEQ ID NO: 49. According to one embodiment, the light chain variable region(s) present in the antigen binding fragment comprise(s) the amino acid sequence of SEQ ID NOs: 50 or an amino acid sequence which is at least 75%, in particular at least 80%, at least 85%, at least 90%, at least 95% or at least 97% identical to one of said sequences. In certain embodiments, the light chain variable region of the antigen binding fragment comprises an amino acid sequence (i) which comprises a set of CDRs wherein CDR-L1 has the amino acid sequence of SEQ ID NO: 47, CDR-L2 has the amino acid sequence of SEQ ID NO: 48 and CDR-L3 has the amino acid sequence of SEQ ID NO: 49 and (ii) which is at least 80%, at least 85%, at least 90%, or at least 95% identical to any one of SEQ ID NOs: 50.

In particular preferred embodiments, the antigen binding fragment comprises at least one, in particular one, heavy chain variable region comprising the amino acid sequence of SEQ ID NO: 46 and at least one, in particular one, light chain variable region comprising the amino acid sequence of SEQ ID NO: 50. In a further embodiment, the antibody module is derived from an antibody comprising one or more of the sequences described above.

In further embodiments, the antigen binding fragment may be derived from any anti-CD3 antibody. Suitable examples of anti-CD3 antibodies from which the antigen binding fragment may be derived include OKT3, TR66, UCHT1, CLB-T3, L2K, DiL2K and the anti-CD3 antibodies disclosed in WO 2008/119567 A2. In certain embodiments, the antigen binding fragment comprises a heavy chain variable region which amino acid sequence is at least 85%, in particular at least 90% or at least 95%, especially 100% identical to the heavy chain variable region of any one of these anti-CD3 antibodies. Preferably, the antigen binding fragment further comprises a light chain variable region which amino acid sequence is at least 85%, in particular at least 90% or at least 95%, especially 100% identical to the light chain variable region of the same anti-CD3 antibodies.

The Multispecific Antibody Construct

The multispecific antibody construct comprises at least one anti-MUC1 antibody module specifically and at least one anti-CD3 antigen binding fragment. In certain embodiments, the multispecific antibody construct comprises at least two, in particular exactly two anti-CD3 antigen binding fragments. The antigen binding fragments may be identical or different and in particular have the same amino acid sequence. At least one of the antigen binding fragments is fused to the C terminus of a heavy chain of the antibody module.

In specific embodiments wherein the multispecific antibody construct comprises two antigen binding fragments, the antibody module also comprises two heavy chains and each of the antigen binding fragments is fused to the C terminus of a different heavy chain of the antibody module. In certain embodiments, an antigen binding fragment is fused to the C terminus of a heavy chain of the antibody module and a further antigen binding fragment is fused to the C terminus of the first antigen binding fragment. If the antibody module comprises two heavy chains, this may be the case on both of these heavy chains.

The antigen binding fragment may be fused to the C terminus of the heavy chain of the antibody module directly via a peptide bond or indirectly via a peptide linker. A direct fusion refers to embodiments wherein the sequence of the antigen binding fragment directly follows the sequence of the heavy chain of the antibody module without any intermediate amino acids between these two sequences. A fusion via a peptide linker refers to embodiments wherein one or more amino acids are present between the sequence of the heavy chain of the antibody module and the sequence of the antigen binding fragment. These one or more amino acids form the peptide linker between the heavy chain of the antibody module and the antigen binding fragment.

It was found that stability of the multispecific antibody construct can be improved by specifically designing the attachment of the antigen binding fragment to the C terminus of the heavy chain of the antibody module. Especially, under certain stress conditions the fusion polypeptide comprising the heavy chain of the antibody module and the antigen binding fragment may degenerate and the antigen binding fragment may be cleaved off from the heavy chain of the antibody module. This can be prevented by mutation of the C terminus of the heavy chain, as demonstrated in the examples. In certain embodiments, in particular in embodiments where the antibody module is an IgG-type antibody module and/or has a γ-type heavy chain, the C terminal amino acid residue of the heavy chain of the antibody module is not a lysine residue. Generally, the γ-type heavy chain of IgG antibodies has a lysine residue as last amino acid at the C terminus. Substitution or deletion of this lysine residue inhibits degradation of the fusion polypeptide. In specific embodiments, the lysine residue at the C terminus of the γ-type heavy chain of the antibody module is deleted or substituted by another amino acid, preferably deleted.

The peptide linker may in principle have any number of amino acids and any amino acid sequence which are suitable for linking the heavy chain of the antibody module and the antigen binding fragment. In certain embodiments, the peptide linker comprises at least 3, preferably at least 5, at least 8, at least 10, at least 15 or at least 20 amino acids. In further embodiments, the peptide linker comprises 50 or less, preferably 45 or less, 40 or less, 35 or less, 30 or less, 25 or less or 20 or less amino acids. In particular, the peptide linker comprises from 5 to 25 amino acids, especially from 10 to 20 amino acids. In specific embodiments, the peptide linker consists of glycine and serine residues. Glycine and serine may be present in the peptide linker in a ratio of 2 to 1, 3 to 1, 4 to 1 or 5 to 1 (number of glycine residues to number of serine residues). For example, the peptide linker may comprise a sequence of four glycine residues followed by one serine residue, and in particular 1, 2, 3, 4, 5 or 6 repeats of this sequence. Specific examples are peptide linkers comprising or consisting of 2 repeats of the amino acid sequence GGGGS (SEQ ID NO: 51), 3 repeats of the amino acid sequence GGGGS (SEQ ID NO: 51) and 4 repeats of the amino acid sequence GGGGS (SEQ ID NO: 51). Especially peptide linkers consisting of 3 or 4 repeats of the amino acid sequence GGGGS (SEQ ID NO: 51) may be used. In specific embodiments, the multispecific antibody construct comprises a peptide linker comprising 3 or 4 repeats of the amino acid sequence GGGGS (SEQ ID NO: 51) between the C terminus of the heavy chains of the antibody module and the N terminus of the antigen binding fragments and/or a peptide linker comprising 3 or 4 repeats of the amino acid sequence GGGGS (SEQ ID NO: 51) between the VH domain and the VL domain of the antigen binding fragments. In other embodiments the peptide linker comprises sequences which show no or only minor immunogenic potential in humans, preferably sequences which are human sequences or naturally occurring sequences. In a further preferred embodiment the peptide linker and the adjacent amino acids show no or only minor immunogenic potential. Peptide linkers as described above may also be used to link other elements of the multispecific antibody construct, such as a heavy chain variable region and a light chain variable region present in one antigen binding fragment.

The multispecific antibody construct in particular is a bispecific antibody construct. The bispecific antibody construct specifically binds to an epitope of MUC1 and to an epitope of CD3, but does not comprise any further antigen binding sites specifically binding to another antigen. In alternative embodiments, the multispecific antibody construct comprises one or more further antigen binding sites specifically binding other antigens. These further antigen bindings sites may be present anywhere in the multispecific antibody construct. In certain embodiments, a further antigen binding site is fused to the C terminus of an antibody light chain of the antibody module. In particular, if the antibody module comprises two antibody light chains, one or more antigen binding sites, especially one antigen binding site, are fused to the C terminus of each of the antibody light chains of the antibody module. These antigen binding sites may be identical or different, and in particular have the same amino acid sequence. The additional antigen binding sites may specifically bind to any antigen, especially to tumor-associated antigens or checkpoint antigens of immune cells. Suitable examples of such antigens may be selected from the group consisting of EGFR, HER2, PD-1, PD-L1, CD40, CEA, EpCAM, CD7, CD28, GITR, ICOS, OX40, 4-1BB, CTLA-4, TFα, LeY, CD160, Galectin-3, Galectin-1.

In certain embodiments, the multispecific antibody construct comprises one or more further agents conjugated thereto. The further agent may be any agent suitable for conjugation to the multispecific antibody construct. If more than one further agent is present in the multispecific antibody construct, these further agents may be identical or different, and in particular are all identical. Conjugation of the further agent to the multispecific antibody construct can be achieved using any methods known in the art. The further agent may be covalently, in particular by fusion or chemical coupling, or non-covalently attached to the multispecific antibody construct. In certain embodiments, the further agent is covalently attached to the multispecific antibody construct, especially via a linker moiety. The linker moiety may be any chemical entity suitable for attaching the further agent to the multispecific antibody construct.

In certain embodiments, the further agent is a polypeptide of protein. This polypeptide or protein may in particular be fused to a polypeptide chain of the antibody module or a polypeptide chain of the antigen binding fragment. In certain embodiments, the further agent being a polypeptide or protein is fused to the C terminus of an antibody light chain of the antibody module. In embodiments wherein the antibody module comprises two antibody light chains, a further agent being a polypeptide or protein may be fused to the C terminus of each of the two antibody light chains. The polypeptide or protein may be identical or different and in particular have the same amino acid sequence. Suitable examples of such further agents being a polypeptide or protein may be selected from the group consisting of cytokines, chemokines, antibody modules, antigen binding fragments, enzymes, and interaction domains.

The further agent preferably is useful in therapy, diagnosis, prognosis and/or monitoring of a disease, in particular cancer. For example, the further agent may be selected from the group consisting of radionuclides, chemotherapeutic agents, detectable labels, toxins, cytolytic components, immunomodulators, immunoeffectors, and liposomes.

Glycosylation of the Multispecific Antibody Construct

The anti-MUC1 antibody module may comprise a CH2 domain in one or more antibody heavy chains. Natural human antibodies of the IgG type comprise an N-glycosylation site in the CH2 domain. The CH2 domains present in the antibody module may or may not comprise an N-glycosylation site.

In certain embodiments, the CH2 domains present in the antibody module do not comprise an N-glycosylation site. In particular, the antibody module does not comprise an asparagine residue at the position in the heavy chain corresponding to position 297 according to the IMGT/Eu numbering system. For example, the antibody module may comprise an Ala297 mutation in the heavy chain. In these embodiments, the multispecific antibody construct preferably has a strongly reduced ability or completely lacks the ability to induce, via binding to Fcγ receptors, antibody-dependent cellular cytotoxicity (ADCC) and/or antibody-dependent cellular phagocytosis (ADCP) and/or complement-dependent cytotoxicity (CDC). Strongly reduced ability in this respect in particular refers to a reduction to 10% or less, especially 3% or less, 1% or less or 0.1% or less activity compared to the same multispecific antibody construct comprising an N-glycosylation site in its CH2 domains and having a common mammalian glycosylation pattern such as those obtainable by production in human cell lines or in CHO cell lines, for example a glycosylation pattern as described herein.

It was surprisingly found that the multispecific antibody constructs which do not comprise an N-glycosylation site in the CH2 domains of the antibody module nevertheless are capable of activating natural killer cells (NK cells) although these constructs are not capable of binding to the Fcγ receptors of NK cells. It is believed that the multispecific antibody constructs recruit and activate T cells, which in turn activate NK cells.

In alternative embodiments, the CH2 domains present in the antibody module comprise an N-glycosylation site. This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. Preferably, the glycans attached to the antibody module are biantennary complex type N-linked carbohydrate structures, preferably comprising at least the following structure:

Asn-GlcNAc-GlcNAc-Man-(Man-GlcNAc)₂

wherein Asn is the asparagine residue of the polypeptide portion of the antibody module; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn.

In preferred embodiments, the multispecific antibody construct does not comprise N-glycolyl neuraminic acids (NeuGc) or detectable amounts of NeuGc. Furthermore, the multispecific antibody construct preferably also does not comprise Galili epitopes (Galα1,3-Gal structures) or detectable amounts of the Galili epitope. In particular, the relative amount of glycans carrying NeuGc and/or Galα1,3-Gal structures is less than 0.1% or even less than 0.02% of the total amount of glycans attached to the CH2 domains of the antibody modules in the population multispecific antibody constructs.

In particular, the multispecific antibody construct has a human glycosylation pattern. Due to these glycosylation properties, foreign immunogenic non-human structures which induce side effects are absent which means that unwanted side effects or disadvantages known to be caused by certain foreign sugar structures such as the immunogenic non-human sialic acids (NeuGc) or the Galili epitope (Gal-Gal structures), both known for rodent production systems, or other structures like immunogenic high-mannose structures as known from e.g. yeast systems are avoided.

In specific embodiments, the multispecific antibody construct comprises a glycosylation pattern at the CH2 domains of the antibody module having a detectable amount of glycans carrying a bisecting GlcNAc residue. In particular, the relative amount of glycans carrying a bisecting GlcNAc residue is at least 0.5%, especially at least 1% of the total amount of glycans attached to the glycosylation sites of the CH2 domains of the antibody module in a composition. Furthermore, in certain embodiments the glycosylation pattern at the CH2 domains comprises a relative amount of glycans carrying at least one galactose residue of at least 30% of the total amount of glycans attached to the CH2 domains of the antibody module in a composition. In particular, the relative amount of glycans carrying at least one galactose residue is at least 40%, especially at least 45% or at least 50% of the total amount of glycans attached to the CH2 domains of the antibody module in a composition.

The multispecific antibody construct may have a glycosylation pattern at the CH2 domains of the antibody module having a high amount of core fucose or a low amount of core fucose. A reduced amount of fucosylation at the CH2 domains increases the ability of the multispecific antibody construct to induce ADCC. In certain embodiments, the relative amount of glycans carrying a core fucose residue is 40% or less, especially 30% or less or 20% or less of the total amount of glycans attached to the CH2 domains of the antibody module in a composition. In alternative embodiments, the relative amount of glycans carrying a core fucose residue is at least 60%, especially at least 65% or at least 70% of the total amount of glycans attached to the CH2 domains of the antibody module in a composition.

Via the presence or absence of the glycosylation site in the CH2 domain of the anti-MUC1 antibody module and the presence or absence of fucose in the glycan structures at said glycosylation site, the ability of the multispecific antibody construct to induce ADCC via the Fc part of the antibody module and the strength of said ADCC induction can be controlled. ADCC mediated by cytotoxic T lymphocytes is already initiated by recruitment of said T lymphocytes to the tumor site. This is achieved by the two binding sites of the multispecific antibody construct, the anti-MUC1 antibody module binding to the tumor cells and the anti-CD3 antigen binding fragment binding to the cytotoxic T lymphocytes. The overall ADCC activity as mediated by T cells and NK cells may be increased by glycosylation of the Fc part of the antibody module and further by reducing the amount of fucosylation in said glycosylation. With Fc-glycosylation, in particular with low fucosylation, ADCC mediated by NK cells is added to the ADCC mediated by T cells. In certain applications, fine tuning of the ADCC activity is important. Therefore, in certain situations, the multispecific antibody construct without a glycosylation site in the CH2 domain of the antibody module, the multispecific antibody construct with a glycosylation site in the CH2 domain of the antibody module and with a high amount of fucosylation, or the multispecific antibody construct with a glycosylation site in the CH2 domain of the antibody module and with a low amount of fucosylation may be most advantageous.

The multispecific antibody construct is preferably recombinantly produced in a host cell. The host cell used for the production of the multispecific antibody construct may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In certain embodiments, the multispecific antibody construct is produced recombinantly in a human blood cell line, in particular in a human myeloid leukemia cell line. Preferred human cell lines which can be used for production of the multispecific antibody construct as well as suitable production procedures are described in WO 2008/028686 A2. In a specific embodiment, the multispecific antibody construct is obtained by expression in a human myeloid leukemia cell line selected from the group consisting of NM-H9D8, NM-H9D8-E6 and NM-H9D8-E6Q12. These cell lines were deposited under the accession numbers DSM ACC2806 (NM-H9D8; deposited on Sep. 15, 2006), DSM ACC2807 (NM-H9D8-E6; deposited on Oct. 5, 2006) and DSM ACC2856 (NM-H9D8-E6Q12; deposited on Aug. 8, 2007) according to the requirements of the Budapest Treaty at the Deutsche Sammlung von Mikroorganismen und Zellkulturen (DSMZ), Inhoffenstraße 7B, 38124 Braunschweig (DE) by Glycotope GmbH, Robert-Rössle-Str. 10, 13125 Berlin (DE). NM-H9D8 cells provide a glycosylation pattern with a high degree of sialylation, a high degree of bisecting GlycNAc, a high degree of galactosylation and a high degree of fucosylation. NM-H9D8-E6 and NM-H9D8-E6Q12 cells provide a glycosylation pattern similar to that of NM-H9D8 cells, except that the degree of fucosylation is very low. Other suitable cell lines include K562, a human myeloid leukemia cell line present in the American Type Culture Collection (ATCC CCL-243), as well as cell lines derived from the aforementioned.

The Nucleic Acid, Expression Cassette, Vector, Cell Line and Composition

In a further aspect, the present invention provides a nucleic acid encoding the multispecific antibody construct. The nucleic acid sequence of said nucleic acid may have any nucleotide sequence suitable for encoding the multispecific antibody construct. However, preferably the nucleic acid sequence is at least partially adapted to the specific codon usage of the host cell or organism in which the nucleic acid is to be expressed, in particular the human codon usage. The nucleic acid may be double-stranded or single-stranded DNA or RNA, preferably double-stranded DNA such as cDNA or single-stranded RNA such as mRNA. It may be one consecutive nucleic acid molecule or it may be composed of several nucleic acid molecules, each coding for a different part of the multispecific antibody construct.

If the multispecific antibody construct is composed of more than one different amino acid chain, such as a light chain and a heavy chain of the antibody module, the nucleic acid may, for example, be a single nucleic acid molecule containing several coding regions each coding for one of the amino acid chains of the multispecific antibody construct, preferably separated by regulatory elements such as IRES elements in order to generate separate amino acid chains, or the nucleic acid may be composed of several nucleic acid molecules wherein each nucleic acid molecule comprises one or more coding regions each coding for one of the amino acid chains of the multispecific antibody construct. In addition to the coding regions encoding the multispecific antibody construct, the nucleic acid may also comprise further nucleic acid sequences or other modifications which, for example, may code for other proteins, may influence the transcription and/or translation of the coding region(s), may influence the stability or other physical or chemical properties of the nucleic acid, or may have no function at all.

In a further aspect, the present invention provides an expression cassette or vector comprising a nucleic acid according to the invention and a promoter operatively connected with said nucleic acid. In addition, the expression cassette or vector may comprise further elements, in particular elements which are capable of influencing and/or regulating the transcription and/or translation of the nucleic acid, the amplification and/or reproduction of the expression cassette or vector, the integration of the expression cassette or vector into the genome of a host cell, and/or the copy number of the expression cassette or vector in a host cell. Suitable expression cassettes and vectors comprising respective expression cassettes for expressing antibodies are well known in the prior art and thus, need no further description here.

Furthermore, the present invention provides a host cell comprising the nucleic acid according to the invention or the expression cassette or vector according to the invention. The host cell may be any host cell. It may be an isolated cell or a cell comprised in a tissue. Preferably, the host cell is a cultured cell, in particular a primary cell or a cell of an established cell line, preferably a tumor-derived cell. Preferably, it is a bacterial cell such as E. coli, a yeast cell such as a Saccharomyces cell, in particular S. cerevisiae, an insect cell such as a Sf9 cell, or a mammalian cell, in particular a human cell such as a tumor-derived human cell, a hamster cell such as CHO, or a primate cell. In a preferred embodiment of the invention the host cell is derived from human myeloid leukaemia cells. Preferably, it is selected from the following cells or cell lines: K562, KG1, MUTZ-3 or a cell or cell line derived therefrom, or a mixture of cells or cell lines comprising at least one of those aforementioned cells. The host cell is preferably selected from the group consisting of NM-H9D8, NM-H9D8-E6, NM H9D8-E6Q12, and a cell or cell line derived from anyone of said host cells, or a mixture of cells or cell lines comprising at least one of those aforementioned cells. These cell lines and their properties are described in detail in the PCT-application WO 2008/028686 A2. In preferred embodiments, the host cell is optimized for expression of glycoproteins, in particular antibodies, having a specific glycosylation pattern. Preferably, the codon usage in the coding region of the nucleic acid according to the invention and/or the promoter and the further elements of the expression cassette or vector are compatible with and, more preferably, optimized for the type of host cell used. Preferably, the multispecific antibody construct is produced by a host cell or cell line as described above.

In another aspect, the present invention provides a composition comprising the multispecific antibody construct, the nucleic acid, the expression cassette or vector, or the host cell. The composition may also contain more than one of these components. Furthermore, the composition may comprise one or more further components selected from the group consisting of solvents, diluents, and excipients Preferably, the composition is a pharmaceutical composition. In this embodiment, the components of the composition preferably are all pharmaceutically acceptable. The composition may be a solid or fluid composition, in particular a—preferably aqueous—solution, emulsion or suspension or a lyophilized powder.

Use in Medicine

The multispecific antibody construct in particular is useful in medicine, in particular in therapy, diagnosis, prognosis and/or monitoring of a disease, in particular a disease as described herein, preferably cancer, infections, inflammatory diseases, graft-versus-host disease and immunodeficiencies.

Therefore, in a further aspect, the invention provides the multispecific antibody construct, the nucleic acid, the expression cassette or vector, the host cell, or the composition for use in medicine. Preferably, the use in medicine is a use in the treatment, prognosis, diagnosis and/or monitoring of a disease such as, for example, diseases associated with abnormal cell growth such as cancer, infections such as bacterial, viral, fungal or parasitic infections, inflammatory diseases such as autoimmune diseases and inflammatory bowel diseases, and diseases associated with a reduce immune activity such as immunodeficiencies. In a preferred embodiment, the disease is cancer. Preferably the cancer is selected from the group consisting of ovarian cancer, breast cancer such as triple negative breast cancer, lung cancer and pancreatic cancer. The cancer may further in particular be selected from colon cancer, stomach cancer, liver cancer, kidney cancer, bladder cancer, skin cancer, cervix cancer, prostate cancer, gastrointestinal cancer and blood cancer.

In certain embodiments, the viral infection is caused by human immunodeficiency virus, herpes simplex virus, Epstein Barr virus, influenza virus, lymphocytic choriomeningitis virus, hepatitis B virus or hepatitis C virus. The inflammatory disease may be selected from inflammatory bowel disease, pelvic inflammatory disease, ischemic stroke, Alzheimer's disease, asthma, pemphigus vulgaris and dermatitis/eczema. The autoimmune disease may be selected from the group consisting of celiac disease, diabetes mellitus type 1, Graves' disease, inflammatory bowel disease, multiple sclerosis, psoriasis, rheumatoid arthritis, systemic lupus erythematosus, vitiligo, psoriatic arthritis, atopic dermatitis, scleroderma, sarcoidosis, primary biliary cirrhosis, Guillain-Barre syndrome, autoimmune hepatitis and ankylosing spondylitis. In certain embodiments, the disease comprises or is associated with cells which express MUC1. For example, a cancer to be treated is MUC1 positive, i.e. comprises cancer cells which express MUC1.

In specific embodiments, the multispecific antibody construct is used in combination with another therapeutic agent, especially another anti-cancer agent. Said further therapeutic agent may be any known anti-cancer drug. Suitable anti-cancer therapeutic agents which may be combined with the multispecific antibody construct may be chemotherapeutic agents, antibodies, immunostimulatory agents, cytokines, chemokines, and vaccines. Furthermore, therapy with the multispecific antibody construct may be combined with radiation therapy, surgery and/or traditional Chinese medicine

### Specific Embodiments

In the following, specific embodiments of the present invention are described.

### Embodiment 1

A multispecific antibody construct, comprising
(i) an anti-MUC1 antibody module, the antibody module comprising at least one antibody heavy chain, and
(ii) an anti-CD3 antigen binding fragment;
   wherein the antigen binding fragment is fused to the C terminus of the heavy chain of the antibody module.

### Embodiment 2

The multispecific antibody construct according to Embodiment 1, wherein the anti-MUC1 antibody module comprises two heavy chains, each comprising a VH domain, a CH1 domain, a hinge region, a CH2 domain and a CH3 domain.

### Embodiment 3

The multispecific antibody construct according to Embodiment 1 or 2, wherein the anti-MUC1 antibody module comprises two light chains, each comprising a VL domain and a CL domain.

### Embodiment 4

The multispecific antibody construct according to any one of Embodiments 1 to 3, wherein the anti-MUC1 antibody module is an IgG-type antibody module, in particular an IgG1-type antibody module, which optionally does not have a lysine residue at the C terminus of the heavy chain.

### Embodiment 5

The multispecific antibody construct according to any one of Embodiments 1 to 4, wherein the anti-MUC1 antibody module has a κ-chain.

### Embodiment 6

The multispecific antibody construct according to any one of Embodiments 1 to 5, wherein the anti-MUC1 antibody module specifically binds to a TA-MUC1 epitope.

### Embodiment 7

The multispecific antibody construct according to Embodiment 6, wherein the anti-MUC1 antibody module comprises a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5, or CDR-H1 having the amino acid sequence of SEQ ID NO: 2, CDR-H2 having the amino acid sequence of SEQ ID NO: 4 and CDR-H3 having the amino acid sequence of SEQ ID NO: 6.

### Embodiment 8

The multispecific antibody construct according to Embodiment 6 or 7, wherein the anti-MUC1 antibody module comprises an antibody heavy chain variable region sequence which is at least 80% identical to any one of SEQ ID NOs: 7, 8 and 9.

### Embodiment 9

The multispecific antibody construct according to any one of Embodiments 1 to 5, wherein the anti-MUC1 antibody module specifically binds to TFα.

### Embodiment 10

The multispecific antibody construct according to Embodiment 9, wherein the anti-MUC1 antibody module comprises a set of heavy chain CDR sequences with CDR-H1 having the amino acid sequence of SEQ ID NO: 21, CDR-H2 having the amino acid sequence of SEQ ID NO: 22 or 23 and CDR-H3 having the amino acid sequence of SEQ ID NO: 24, 25 or 26.

### Embodiment 11

The multispecific antibody construct according to Embodiment 9 or 10, wherein the anti-MUC1 antibody module comprises an antibody heavy chain variable region sequence which is at least 80% identical to any one of SEQ ID NOs: 27 to 32.

### Embodiment 12

The multispecific antibody construct according to any one of Embodiments 1 to 11, wherein the anti-CD3 antigen binding fragment comprises at least one VH domain and optionally at least one VL domain.

### Embodiment 13

The multispecific antibody construct according to any one of Embodiments 1 to 12, wherein the anti-CD3 antigen binding fragment does not comprise any antibody constant region domains.

### Embodiment 14

The multispecific antibody construct according to any one of Embodiments 1 to 13, wherein the anti-CD3 antigen binding fragment is a scFv fragment.

### Embodiment 15

The multispecific antibody construct according to any one of Embodiments 1 to 14, wherein the anti-CD3 antigen binding fragment specifically binds to CD3ε.

### Embodiment 16

The multispecific antibody construct according to any one of Embodiments 1 to 15, wherein the anti-CD3 antigen binding fragment comprises a set of heavy chain CDR sequences with CDR-H1 having the amino acid sequence of SEQ ID NO: 43, CDR-H2 having the amino acid sequence of SEQ ID NO: 44 and CDR-H3 having the amino acid sequence of SEQ ID NO: 45.

### Embodiment 17

The multispecific antibody construct according to any one of Embodiments 1 to 16, wherein the anti-CD3 antigen binding fragment comprises an antibody heavy chain variable region sequence which is at least 80% identical to any one of SEQ ID NOs: 46.

### Embodiment 18

The multispecific antibody construct according to any one of Embodiments 1 to 17, wherein the anti-CD3 antigen binding fragment comprises a peptide linker between the VH domain and the VL domain.

### Embodiment 19

The multispecific antibody construct according to any one of Embodiments 1 to 18, wherein the multispecific antibody construct comprises two anti-CD3 antigen binding fragments, each fused to the C terminus of a different heavy chain of the antibody module.

### Embodiment 20

The multispecific antibody construct according to any one of Embodiments 1 to 19, wherein the multispecific antibody construct comprises a peptide linker between the C terminus of the heavy chain of the antibody module and the antigen binding fragment.

### Embodiment 21

The multispecific antibody construct according to any one of Embodiments 1 to 20, wherein the multispecific antibody construct is a bispecific antibody construct.

### Embodiment 22

The multispecific antibody construct according to any one of Embodiments 1 to 21, wherein the antibody module does not comprise an N-glycosylation site in the CH2 domain.

### Embodiment 23

The multispecific antibody construct according to any one of Embodiments 1 to 21, wherein the antibody module comprises an N-glycosylation site in the CH2 domain of the antibody heavy chains.

### Embodiment 24

The multispecific antibody construct according to Embodiment 23, wherein the antibody module has a glycosylation pattern in the CH2 domain of the antibody heavy chains, having one or more of the following characteristics
(i) a relative amount of glycans carrying a bisecting GlcNAc residue of at least 0.5% of the total amount of glycans attached to the glycosylation sites of the CH2 domains of the antibody module in a composition;
(ii) a relative amount of glycans carrying at least one galactose residue of at least 30% of the total amount of glycans attached to the CH2 domains of the antibody module in a composition;
(iii) a relative amount of glycans carrying a core fucose residue of at least 60% of the total amount of glycans attached to the CH2 domains of the antibody module in a composition.

### Embodiment 25

The multispecific antibody construct according to Embodiment 23, wherein the antibody module has a glycosylation pattern in the CH2 domain of the antibody heavy chains, having one or more of the following characteristics
(i) a relative amount of glycans carrying a bisecting GlcNAc residue of at least 0.5% of the total amount of glycans attached to the glycosylation sites of the CH2 domains of the antibody module in a composition;
(ii) a relative amount of glycans carrying at least one galactose residue of at least 30% of the total amount of glycans attached to the CH2 domains of the antibody module in a composition;
(iii) a relative amount of glycans carrying a core fucose residue of 40% or less of the total amount of glycans attached to the CH2 domains of the antibody module in a composition.

### Embodiment 26

The multispecific antibody construct according to any one of Embodiments 1 to 25, comprising a further agent conjugated thereto.

### Embodiment 27

The multispecific antibody construct according to Embodiment 26, wherein the further agent is a polypeptide or protein which is fused to a polypeptide chain of the antibody module or to a polypeptide chain of the antigen binding fragment.

### Embodiment 28

The multispecific antibody construct according to Embodiment 27, wherein the antibody module comprises at least one antibody light chain and the further agent being a polypeptide or protein is fused to the C terminus of said antibody light chain.

### Embodiment 29

The multispecific antibody construct according to any one of Embodiments 26 to 28, wherein the further agent is selected from the group consisting of cytokines, chemokines, antibody modules, antigen binding fragments, enzymes and binding domains.

### Embodiment 30

A nucleic acid encoding the multispecific antibody construct according to any one of Embodiments 1 to 29.

### Embodiment 31

An expression cassette or vector comprising the nucleic acid according to Embodiment 30 and a promoter operatively connected with said nucleic acid.

### Embodiment 32

A host cell comprising the nucleic acid according to Embodiment 30 or the expression cassette or vector according to Embodiment 31.

### Embodiment 33

A pharmaceutical composition comprising the multispecific antibody construct according to any one of Embodiments 1 to 29 and one or more further components selected from the group consisting of solvents, diluents, and excipients.

### Embodiment 34

The multispecific antibody construct according to any one of Embodiments 1 to 29 or the pharmaceutical composition according to Embodiment 33 for use in medicine.

### Embodiment 35

The multispecific antibody construct according to any one of Embodiments 1 to 29 or the pharmaceutical composition according to Embodiment 33 for use in the treatment, prognosis, diagnosis and/or monitoring of diseases associated with abnormal cell growth such as cancer; infections such as bacterial, viral, fungal or parasitic infections; inflammatory diseases such as autoimmune diseases and inflammatory bowel diseases; and diseases associated with a reduce immune activity such as immunodeficiencies.

### Embodiment 36

The multispecific antibody construct or pharmaceutical composition according to Embodiment 35 for use in the treatment of cancer, wherein the cancer is selected from the group consisting of cancer of the breast, colon, stomach, liver, pancreas, kidney, blood, lung, and ovary.

### Embodiment 37

The multispecific antibody construct or pharmaceutical composition according to Embodiment 35 for use in the treatment of infections, wherein the infection is selected from the group consisting of bacterial infections, viral infections, fungal infections and parasitic infections.

### Embodiment 38

The multispecific antibody construct or pharmaceutical composition according to Embodiment 35 for use in the treatment of autoimmune diseases, wherein the autoimmune disease is selected from the group consisting of celiac disease, diabetes mellitus type 1, Graves disease, inflammatory bowel disease, multiple sclerosis, psoriasis, rheumatoid arthritis and systemic lupus erythematosus.

### Embodiment 39

A multispecific antibody construct, comprising
(i) an anti-MUC1 antibody module, the antibody module comprising two human γ-type antibody heavy chains and two human κ-type antibody light chains, each heavy chain comprising a VH domain, a CH1 domain, a hinge region, a CH2 domain and a CH3 domain, and each light chain comprising a VL domain and a CL domain; and
(ii) two anti-CD3 antigen binding fragments, each comprising a VH domain, a peptide linker and a VL domain;
   wherein each the antigen binding fragment is fused via a peptide linker to the C terminus of a different heavy chain of the antibody module.

### Embodiment 40

The multispecific antibody construct according to Embodiment 39, wherein each VH domain of the anti-MUC1 antibody module comprises an amino acid sequence which is at least 80% identical, especially 100% identical, to any one of SEQ ID NOs: 7, 8 and 9, and a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5, or CDR-H1 having the amino acid sequence of SEQ ID NO: 2, CDR-H2 having the amino acid sequence of SEQ ID NO: 4 and CDR-H3 having the amino acid sequence of SEQ ID NO: 6.

### Embodiment 41

The multispecific antibody construct according to Embodiment 40, wherein each VL domain of the anti-MUC1 antibody module comprises an amino acid sequence which is at least 80% identical, especially 100% identical, to any one of SEQ ID NOs: 16, 17 or 18, and a set of heavy chain CDR sequences with CDR-L1 having the amino acid sequence of SEQ ID NO: 10, CDR-L2 having the amino acid sequence of SEQ ID NO: 12 and CDR-L3 having the amino acid sequence of SEQ ID NO: 14, or CDR-L1 having the amino acid sequence of SEQ ID NO: 11, CDR-L2 having the amino acid sequence of SEQ ID NO: 13 and CDR-L3 having the amino acid sequence of SEQ ID NO: 15.

### Embodiment 42

The multispecific antibody construct according to Embodiment 39, wherein each VH domain of the anti-MUC1 antibody module comprises an amino acid sequence which is at least 80% identical, especially 100% identical, to any one of SEQ ID NOs: 27 to 32, and a set of heavy chain CDR sequences with CDR-H1 having the amino acid sequence of SEQ ID NO: 21, CDR-H2 having the amino acid sequence of SEQ ID NO: 22 or 23 and CDR-H3 having the amino acid sequence of SEQ ID NO: 24, 25 or 26.

### Embodiment 43

The multispecific antibody construct according to Embodiment 42, wherein each VL domain of the anti-MUC1 antibody module comprises an amino acid sequence which is at least 80% identical, especially 100% identical, to any one of SEQ ID NOs: 40 to 42, and a set of heavy chain CDR sequences with CDR-L1 having the amino acid sequence of SEQ ID NO: 33, 34 or 35, CDR-L2 having the amino acid sequence of SEQ ID NO: 36 or 37 and CDR-L3 having the amino acid sequence of SEQ ID NO: 38 or 39.

### Embodiment 44

The multispecific antibody construct according to any one of Embodiments 39 to 43, wherein the VH domain of each anti-CD3 antigen binding fragment comprises an amino acid sequence which is at least 80% identical, especially 100% identical, to SEQ ID NOs: 46, and a set of heavy chain CDR sequences with CDR-H1 having the amino acid sequence of SEQ ID NO: 43, CDR-H2 having the amino acid sequence of SEQ ID NO: 44 and CDR-H3 having the amino acid sequence of SEQ ID NO: 45.

### Embodiment 45

The multispecific antibody construct according to Embodiment 44, wherein the VL domain of each anti-CD3 antigen binding fragment comprises an amino acid sequence which is at least 80% identical, especially 100% identical, to SEQ ID NOs: 50, and a set of heavy chain CDR sequences with CDR-L1 having the amino acid sequence of SEQ ID NO: 47, CDR-L2 having the amino acid sequence of SEQ ID NO: 48 and CDR-L3 having the amino acid sequence of SEQ ID NO: 49.

### Embodiment 46

The multispecific antibody construct according to any one of Embodiments 39 to 45, comprising a peptide linker comprising 3 or 4 repeats of the amino acid sequence GGGGS (SEQ ID NO: 51) between the C terminus of the heavy chains of the antibody module and the N terminus of the antigen binding fragments and a peptide linker comprising 3 or 4 repeats of the amino acid sequence GGGGS (SEQ ID NO: 51) between the VH domain and the VL domain of the antigen binding fragments.

### Embodiment 47

The multispecific antibody construct according to any one of Embodiments 39 to 46, wherein the heavy chains of the anti-MUC1 antibody module are γ1-type heavy chains.

### Embodiment 48

The multispecific antibody construct according to any one of Embodiments 39 to 47, wherein the anti-MUC1 antibody module does not comprise a lysine residue at the C terminus of the heavy chain.

### Embodiment 49

The multispecific antibody construct according to any one of Embodiments 39 to 48, wherein the antibody module does not comprise an N-glycosylation site in the CH2 domain of each antibody heavy chains.

### Embodiment 50

The multispecific antibody construct according to Embodiment 49, wherein the antibody module does not comprise an asparagine residue at the position in the heavy chain corresponding to position 297 according to the IMGT/Eu numbering system, in particular comprises an Ala297 mutation in the heavy chain.

### Embodiment 51

The multispecific antibody construct according to any one of Embodiments 39 to 48, wherein the antibody module comprises an N-glycosylation site in the CH2 domain of each antibody heavy chains.

### Embodiment 52

The multispecific antibody construct according to Embodiment 51, wherein the antibody module has a glycosylation pattern in the CH2 domain of the antibody heavy chains, wherein the relative amount of glycans carrying a core fucose residue is at least 60%, especially at least 65% or at least 70% of the total amount of glycans attached to the CH2 domains of the antibody module in a composition of the multispecific antibody construct.

### Embodiment 53

The multispecific antibody construct according to Embodiment 51, wherein the antibody module has a glycosylation pattern in the CH2 domain of the antibody heavy chains, wherein the relative amount of glycans carrying a core fucose residue is 40% or less, especially 30% or less or 20% or less of the total amount of glycans attached to the CH2 domains of the antibody module in a composition of the multispecific antibody construct.

### Embodiment 54

The multispecific antibody construct according to any one of Embodiments 39 to 53 for use in the treatment of diseases associated with abnormal cell growth such as cancer, infections such as bacterial, viral, fungal or parasitic infections, inflammatory diseases, autoimmune diseases, graft-versus-host disease and immunodeficiencies.

### Embodiment 55

The multispecific antibody construct according to any one of Embodiments 39 to 53 for use in the treatment of ovarian cancer, breast cancer such as triple negative breast cancer, lung cancer or pancreatic cancer.

### FIGURES

FIG. 1 shows the bispecific antibody constructs of comprising anti-CD3 scFv fragments attached to the C terminus of the light chain (αMUC1-αCD3-Cκ) or to the C terminus of the heavy chain (αMUC1-αCD3-CH3) of an anti-MUC1 antibody.

FIG. 2 shows activation and proliferation of T cells by the bispecific constructs. PBMCs containing T cells were incubated with target cells with different MUC1 expression levels in the presence of αMUC1-αCD3-CH3-NA. No target cells and the monospecific αMUC1 antibody were used as controls. Activation of T cells was determined by CD69 (A) and CD25 (B) expression and proliferation by the number of divided cells (C).

FIG. 3 shows activation and proliferation of NK cells by the bispecific constructs. PBMCs containing NK cells were incubated with target cells with different MUC1 expression levels in the presence of αMUC1-αCD3-CH3-NA. No target cells and the monospecific αMUC1 antibody were used as controls. Activation of NK cells was determined by CD69 (A) and CD25 (B) expression and proliferation by the number of divided cells (C).

FIG. 4 shows binding of the bispecific constructs to their target antigens. αMUC1-αCD3-CH3 and αMUC1-αCD3-Cκ were incubated with cell lines expressing the respective antigen, but not the antigen of the other specificity of the bispecific construct. Further bispecific constructs against HER2 and CD3 as well as the monospecific antibodies against the respective tumor antigens were used as control. Binding to MUC1 (A), HER2 (B) and CD3 (C) was determined.

FIG. 5 shows T cell mediated ADCC against target cells initiated by the bispecific constructs. Pre-activated T cells (A, B) or PBMCs containing non-pre-activated T cells (C, D) were incubated with αMUC1-αCD3-CH3 and αMUC1-αCD3-Cκ (A, C) or αHER2-αCD3-CH3 and αHER2-αCD3-Cκ (B, D) in the presence of MUC1⁺ or HER2⁺ target cells, respectively. Specific lysis of the target cells depending on the concentration of the bispecific antibody construct was determined. The monospecific antibody against the respective target antigen was used as control.

FIG. 6 shows T cell mediated ADCC against target cells initiated by the bispecific constructs targeting TFα. Pre-activated T cells were incubated with αTFα-αCD3-CH3 in the presence of TFα-MUC1⁺ target cells. Specific lysis of the target cells depending on the concentration of the bispecific antibody construct was determined. The monospecific antibody against TFα was used as control.

FIG. 7 shows binding to CD3+ cells of αMUC1-αCD3-CH3 and a similar bispecific antibody not targeting MUC1.

FIG. 8 shows cytokine release of immune cells stimulated with αMUC1-αCD3-CH3 depending on the presence of target cells. The concentration of IFN-γ, TFN-α and IL-6 in the supernatant of PBMCs after 48h incubation with different concentrations of αMUC1-αCD3-CH3 in the presence or absence of MUC1+ MCF-7 target cells is shown.

FIG. 9 shows T cell activation of αMUC1-αCD3-CH3 depending on the presence of target cells. The percentage of CD4+(A, B) or CD8+(C, D) T cells which are positive for the activation markers CD69 (A, C) and CD25 (B, D) was determined via flow cytometry after incubation with αMUC1-αCD3-CH3 or a control bispecific antibody not targeting MUC1 in the presence or absence of MUC1+ MCF-7 target cells.

FIG. 10 shows T cell recruitment to tumor sites by the αMUC1-αCD3-CH3 antibody. Tissue sections of tumor spheroids cocultured with PBMCs in the presence or absence of αMUC1-αCD3-CH3 are shown (A). T cells are stained in dark color. The number of CD8+ T cells per tumor spheroid depending on the presence of αMUC1-αCD3-CH3 was determined (B).

FIG. 11 shows upregulation of TA-MUC1 in tumor cells treated with αMUC1-αCD3-CH3. Tissue sections of tumor spheroids cultured in presence or absence of αMUC1-αCD3-CH3 are shown. TA-MUC1+ cells are stained in dark color.

FIG. 12 shows stimulation of bystander immune cells by αMUC1-αCD3-CH3. PBMCs were incubated with αMUC1-αCD3-CH3 in the presence or absence of target cells. The percentage of specific immune cells which are positive for their respective activation marker was determined via flow cytometry. (A) B cells, (B) monocytes, (C) natural killer T cells, (D) natural killer cells.

FIG. 13 shows a stability analysis of the bispecific αCD3-CH3 antibodies. The heavy chains of the antibody were isolated and its molecular weight analyzed via UPLC-MS. Upper panel: αMUC1-αCD3-CH3 exposed to heavy stress; middle panel: αMUC1-αCD3-CH3 produced without stress; lower panel: αTFα-αCD3-CH3 with deletion of C terminal lysine of the antibody heavy chain, exposed to heavy stress. Molecular mass of the intact heavy chain with the C terminal αCD3-scFv: about 77 kDa; molecular mass of the heavy chain without the C terminal αCD3-scFv: about 49 kDa.

### EXAMPLES

### Example 1: Production of Bispecific Antibody Constructs Specifically Binding MUC1 and CD3

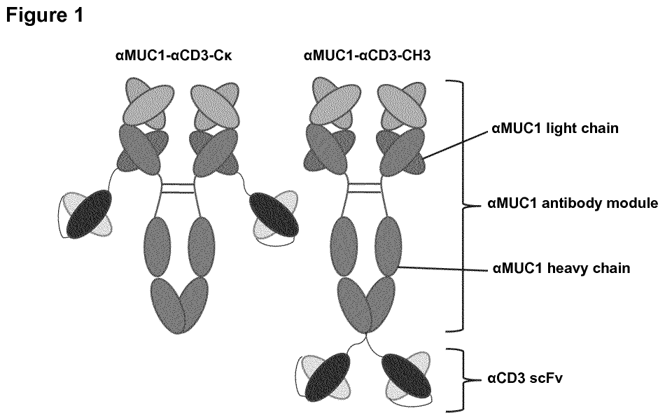

Bispecific T cell engaging constructs were created that consist of a MUC1 specific binding part and a CD3 binding part. The MUC1 binding part is a humanized full-length IgG1 molecule with the typical antibody Y-shape. CD3 binding is realized by fusion of an anti-CD3 single chain variable fragment to each Cκ- (αMUC1-αCD3-Cκ) or CH3-domain (αMUC1-αCD3-CH3) of the αMUC1 IgG. The general structure of the constructs is shown in FIG. 1.

In construct αMUC1-αCD3-CH3-wt, the anti-TA-MUC1 antibody PankoMab (gatipotuzumab) is used as MUC1 binding part and a scFv construct with the variable regions of the anti-CD3 antibody TR66 is used as CD3 binding part. The anti-CD3 scFv is fused to the C terminus of the CH3 domain of PankoMab via a (Gly₄Ser)₄ linker. The construct αMUC1-αCD3-Cκ-wt is identical to αMUC1-αCD3-CH3-wt, except that the anti-CD3 scFv is fused to the C terminus of the Cκ domain of PankoMab.

Constructs αMUC1-αCD3-CH3-NA correspond to the “wt” construct, except for an Asn297Ala mutation in the PankoMab heavy chain which disables the glycosylation site in the CH2 domain. Hence, the “NA” construct is not glycosylated in the Fc part of PankoMab.

The constructs were expressed in the human myeloid leukemia derived cell line NM-H9D8-E6Q12 (DSM ACC2856), producing the constructs with a human glycosylation pattern having a low amount of fucosylation of about 10% in the PankoMab CH2 domain of the wt constructs. Additionally, the αMUC1-αCD3-CH3-wt construct was also produced in the related cell line NM-H9D8 (DSM ACC2806), resulting in glycosylated constructs with a high amount of fucosylation of about 90% in the PankoMab CH2 domain.

Similar constructs were produced using the anti-TFα antibody KaroMab instead of PankoMab as MUC1 binding part.

Production yields using serum-free media were sufficient for subsequent purification, biochemical assessment and bio-functional evaluation of the bispecific antibody mechanisms of action against cancer cell lines. Moreover, to evaluate feasibility to produce large amounts of αMUC1-αCD3-CH3, high producing cell clone was cultivated in a bioreactor under serum-free conditions.

One major challenge of therapeutic bispecific antibodies is the production of sufficient amounts of recombinant protein. To investigate feasibility of high-yield production under conditions compatible with large-scale good manufacturing practice (GMP) production, αMUC1-αCD3-CH3 producing cells were cultivated in a 2 L bioreactor. In the controlled environment using a continuous perfusion process with serum-free medium, high cell densities were reached. Maximal cell concentrations were 3×10⁷ viable cells/mL which is about ten-times higher than maximal cell density in spinner cultures. More importantly, once the culture had reached the maximal perfusion rate, αMUC1-αCD3-CH3 titers reached up to 50 μg/mL and the mean titer for the duration of the perfusion process was 28 μg/mL. Therefore, at a perfusion rate of 2 L/d, the process yielded on average 47 mg αMUC1-αCD3-CH3 per day. The supernatant was harvested over 30 days. Cell viability was above 90%. Cells were removed from the bioreactor by bleeding to avoid cell concentrations exceeding 3×10⁷ viable cells/mL and ensure sufficient nutrient supply for the culture. The total αMUC1-αCD3-CH3 antibody yield was 1.4 g within 30 days cultivation.

αMUC1-αCD3-CH3 was purified by protein A chromatography and analyzed for molecular weight of intact antibody chains using LC-MS. In comparison, αMUC1-αCD3-CH3 was also purified from last supernatant harvested in the end of an exceptional long bioreactor run of more than 40 days.

### Example 2: T Cell Activation and Proliferation

To investigate T cell activation by the bispecific constructs, expression of the activation markers CD69 and CD25 after 48h was analyzed by flow cytometry on CD4⁺ and CD8⁺ T cells. For this purpose human PBMCs from healthy donors were incubated with αMUC1-αCD3-CH3-NA or αMUC1 as control at the indicated concentrations for 48 h. After 48 h PBMC's were harvested and stained with fluorescence labelled αCD45, αCD4, αCD8, αCD25, αCD69, αCD56, αCD14, and αCD19 antibodies, respectively. To solely analyze viable cells DAPI (Sigma-Aldrich) was used. Cells were analyzed in an Attune N×T (Thermo Fisher) flow cytometer. T cell activation was analyzed in absence or presence of cell lines with different levels of MUC1 expression at a 5:1 ratio between effector and target cells.

Besides T cell activation another mechanism of action of T cell recruiting bispecifics is the induction of T cell proliferation. To measure T cell proliferation PBMCs from healthy donors were labeled with CellTrace™ Violet (Thermo Fisher) and incubated with αMUC1-αCD3-CH3-NA or αMUC1 as control in the absence or presence of cell lines with different levels of MUC1 expression at a 5:1 ratio between effector and target cells for 5 days. If T cells proliferate the CellTrace™ dye is diluted for each generation of proliferating cells. After 5 days PBMC's were harvested and stained with fluorescence labelled αCD45, αCD4, αCD8, αCD56, αCD14, and αCD19 antibodies, respectively. To solely analyze viable cells 7-AAD (Calbiochem) was used. Cells were analyzed in an Attune N×T (Thermo Fisher) flow cytometer.

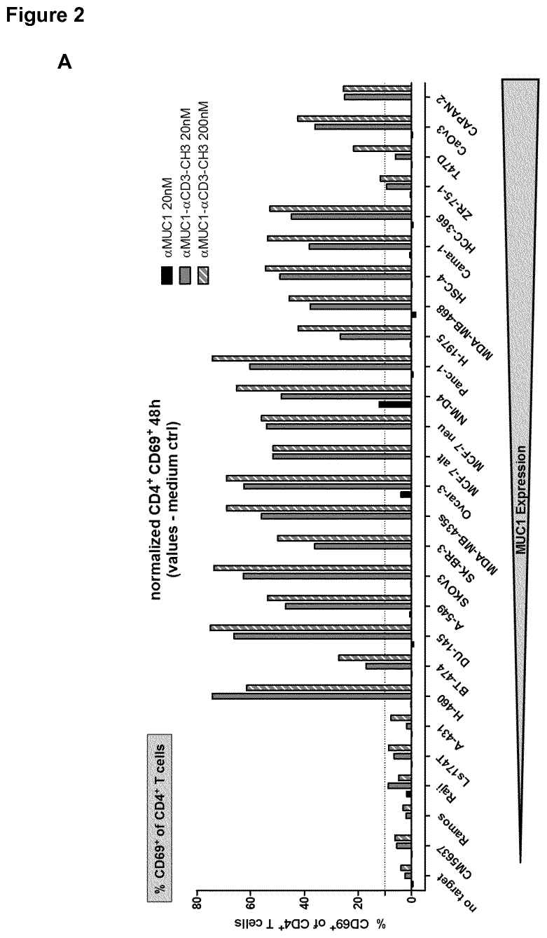

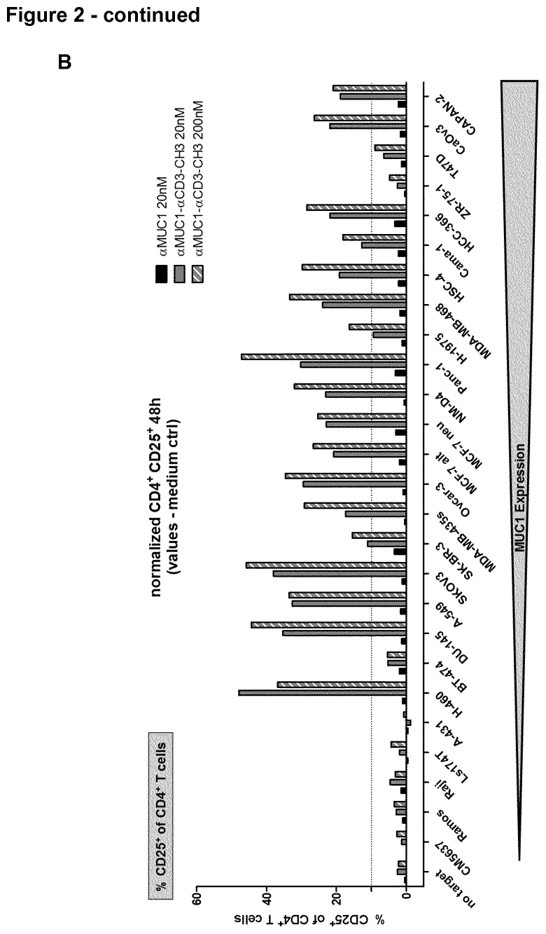

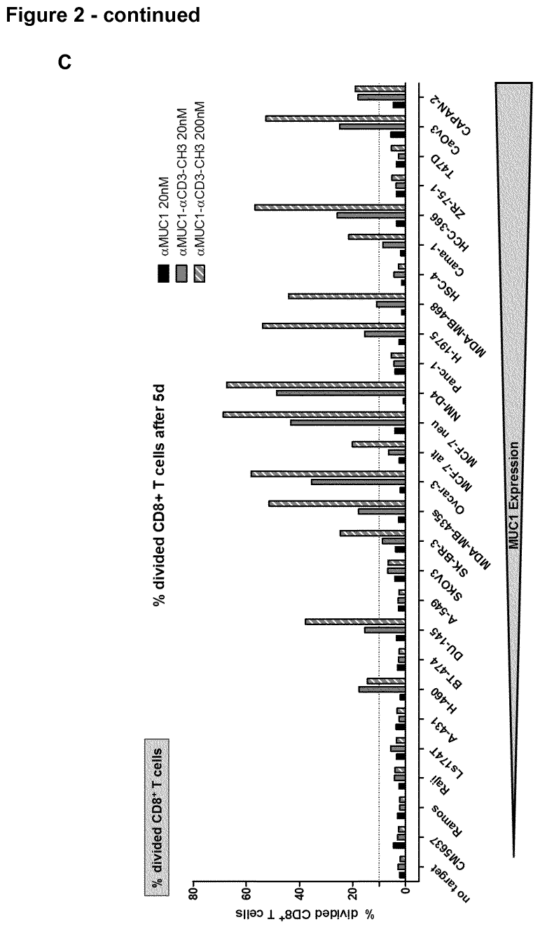

The results demonstrate that αMUC1-αCD3-CH3 is able to induce T cell activation and T cell proliferation by simultaneous binding of MUC1+ cell lines and CD3 on T cells. As shown for CD4+ T cells, incubation with multiple MUC1+ cell lines and αMUC1-αCD3-CH3 leads to increased expression of T cell activation markers CD69 (FIG. 2A) and CD25 (FIG. 2B). Additionally, as shown for CD8+ T cells due to simultaneous binding of MUC1+ cell lines and CD3 on T cells T cell proliferation is induced (FIG. 2C).

### Example 3: NK Cell Activation and Proliferation

To investigate NK cell activation expression of the activation markers CD69 and CD25 after 48h was analyzed by flow cytometry. For this purpose human PBMCs from healthy donors were incubated with αMUC1-αCD3-CH3-NA at the indicated concentrations for 48 h. After 48 h PBMC's were harvested and stained with fluorescence labelled αCD45, αCD4, αCD8, αCD25, αCD69, αCD56, αCD14, and αCD19 antibodies, respectively. To solely analyze viable cells DAPI (Sigma-Aldrich) was used. Cells were analyzed in an Attune N×T (Thermo Fisher) flow cytometer. NK cell activation was analyzed in absence or presence of cell lines with different levels of MUC1 expression at a 5:1 ratio between effector and target cells.

If NK cells are activated they start to proliferate. To measure NK cell proliferation PBMCs from healthy donors were labeled with CellTrace™ Violet (Thermo Fisher) and incubated with αMUC1-αCD3-CH3-NA or αMUC1 as control in the absence or presence of cell lines with different levels of MUC1 expression at a 5:1 ratio between effector and target cells for 5 days. If NK cells proliferate the CellTrace™ dye is diluted for each generation of proliferating cells. After 5 days PBMC's were harvested and stained with fluorescence labelled αCD45, αCD4, αCD8, αCD56, αCD14, and αCD19 antibodies, respectively. To solely analyze viable cells 7-AAD (Calbiochem) was used. Cells were analyzed in an Attune N×T (Thermo Fisher) flow cytometer.

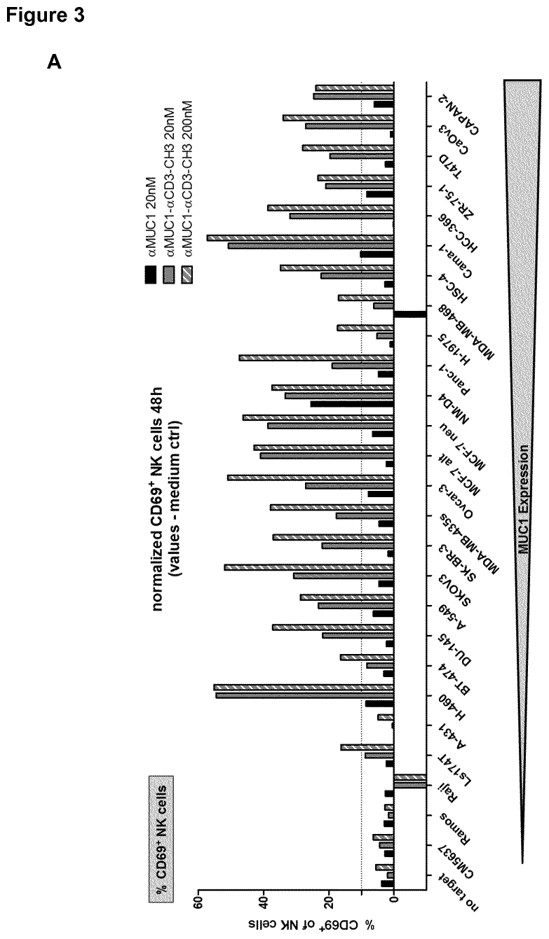

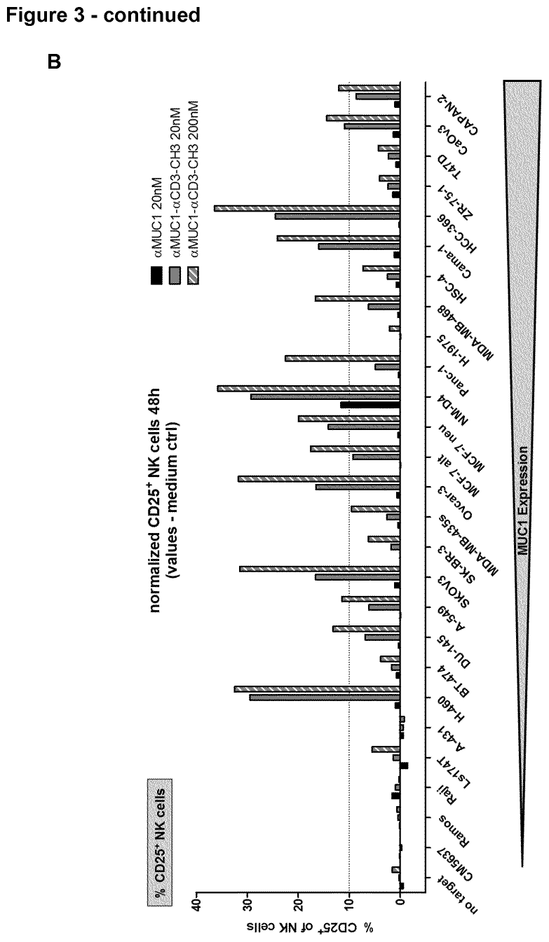

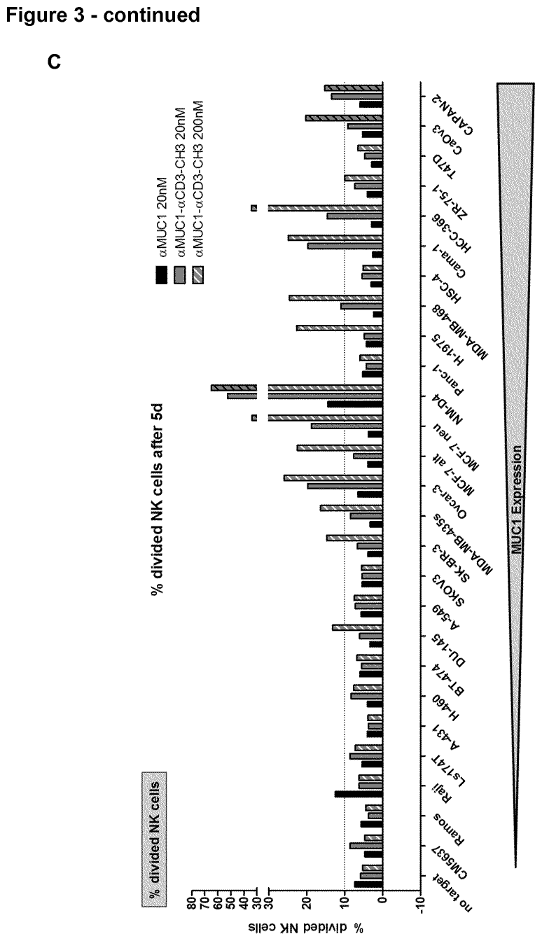

The results demonstrate that αMUC1-αCD3-CH3-NA is able to induce NK cell activation and NK cell proliferation. αMUC1-αCD3-CH3-NA without an N-Glykan at Asn297 is only able to bind T cells via the αCD3 binding moiety and shows no NK cell binding. Surprisingly NK cell activation and proliferation was observed, that depends on the presence of MUC1+ cell lines and αMUC1-αCD3-CH3-NA. Probably due to αMUC1-αCD3-CH3 mediated activation of T cells and subsequent release of stimulatory cytokines an additional activation of NK cells is induced. Therefore incubation with αMUC1-αCD3-CH3-NA, despite no direct NK cell binding, leads to NK cell activation, as shown by up-regulation of activation markers CD69 (FIG. 3A) and CD25 (FIG. 3B) as well as NK cell proliferation (FIG. 3C).

### Example 4: Binding of the Specific Antigens

The binding properties of αMUC1, αMUC1-αCD3-Cκ and αMUC1-αCD3-CH3 as well as αHER2, αHER2-αCD3-Cκ and αHER2-αCD3-CH3 to human TA-MUC1 and HER2 expressing tumor cells were analyzed by flow cytometry. The breast cancer cell line ZR-75-1 with strong TA-MUC1 and medium HER2 expression, but absent CD3 expression was used to determine TA-MUC1 and HER2 binding. To analyze CD3 binding CD3⁺ but TA-MUC1 and Her2 negative Jurkat cells were used. Briefly, target cells were harvested and incubated with indicated antibodies in serial dilutions.

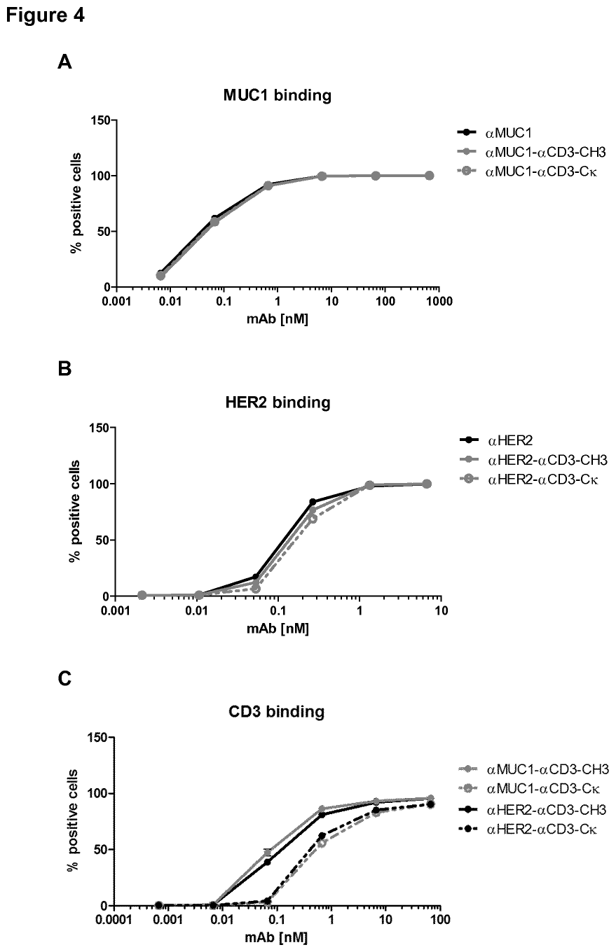

Afterwards, cells were washed and incubated with a secondary goat anti-hlgG (H+L)-RPE-conjugated antibody at 4° C. in the dark. Cells were analyzed via flow cytometry in a FACS Canto II (Becton Dickinson) flow cytometer. αMUC1, αMUC1-αCD3-Cκ and αMUC1-αCD3-CH3 showed comparable binding of TA-MUC1+ cell line ZR-75-1 (FIG. 4A). Neither the addition of αCD3 scFv's to the Cκ-domain nor to the CH3-domain influences binding of TA-MUC1. Similar results were obtained with HER2 targeting constructs, as αHER2, αHER2-αCD3-Cκ and αHER2-αCD3-CH3 showed comparable binding of HER2+ cell line ZR-75-1 (FIG. 4B). In contrast, analyzing the binding to CD3⁺ Jurkat cells αMUC1-αCD3-CH3 as well as αHER2-αCD3-CH3 showed increased binding of CD3 compared to their respective Cκ-fused counterpart αMUC1-αCD3-Cκ and αHER2-αCD3-Cκ (FIG. 4C).

### Example 5: ADCC Assays

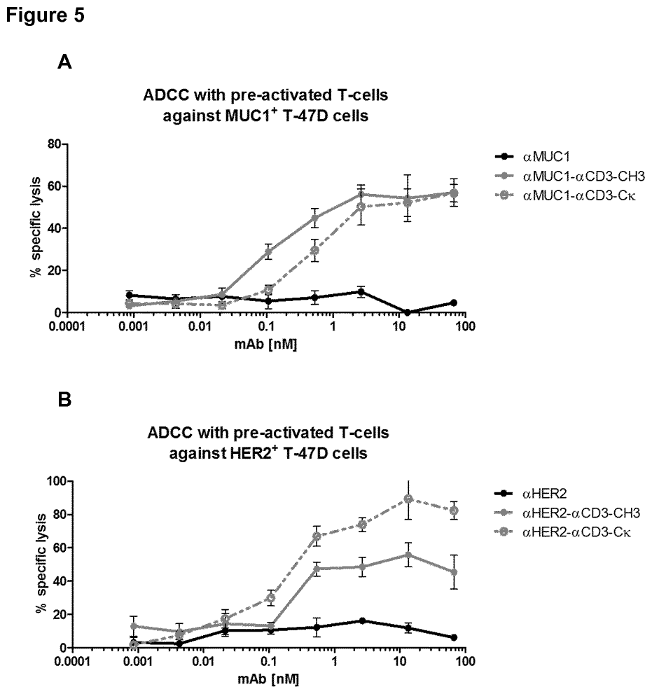

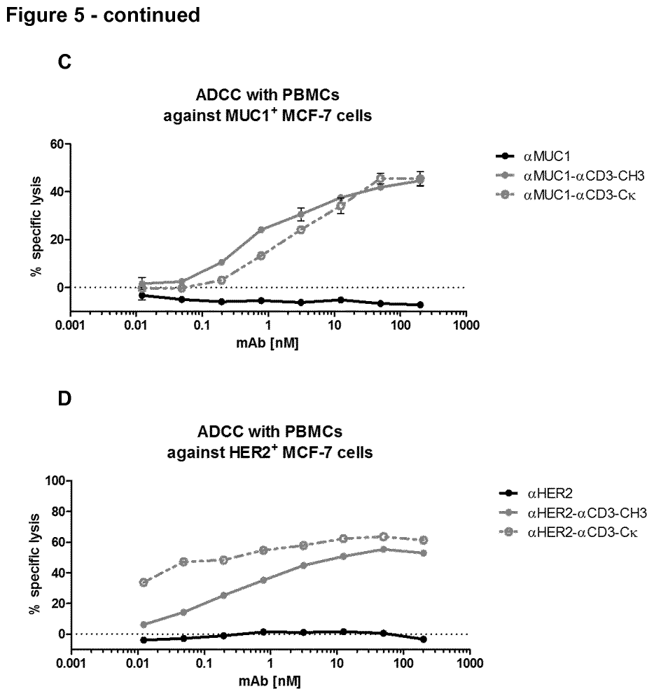

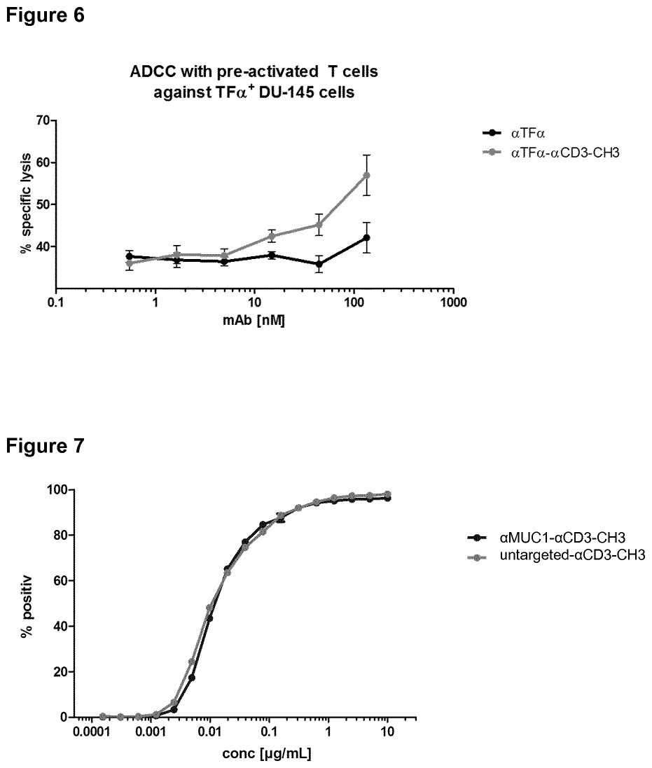

T cell mediated antibody-dependent cell cytotoxicity (ADCC) is a main mechanism of T cell engaging antibodies. After TA-MUC1 or HER2 binding, the bispecific antibody recruits T cells by binding to CD3 on their surface, resulting in the release of cytotoxic granules containing perforin and granzymes that promote cell death of the TA-MUC1′ or HER2⁺ tumor cell. Using pre-activated T cells as effector cells αMUC1-αCD3-Cκ and αMUC1-αCD3-CH3 (FIG. 5A) as well as αHER2-αCD3-Cκ and αHER2-αCD3-CH3 (FIG. 5B) showed effective lysis of TA-MUC1⁺ and HER2⁺ breast cancer cell line T-47D. Surprisingly T cell ADCC mediated by αMUC1-αCD3-CH3 was enhanced compared with αMUC1-αCD3-Cκ (FIG. 5A) whereas αHER2-αCD3-Cκ showed stronger T cell ADCC activity compared to αHER2-αCD3-CH3 (FIG. 5B). The results obtained with the HER2 binding constructs are in line with recent reports that strongly favor short distances between the T cell binding domain and the cancer cell binding domain, as a closer proximity of tumor cell membrane and T cell is described to enhance T cell activation and mediated cytotoxicity (see, e.g., Bluemel, C. et al. (2010) Cancer Immunol Immunother 59:1197-1209; Chames, P. and Baty, D. (2009) mAbs 1(6):539-547). In contrast to this, the distance between the binding domains of αMUC1-αCD3-CH3 wherein the anti-CD3 binding fragment is attached to the C terminus of the heavy chain is much larger compared to αMUC1-αCD3-Cκ wherein the anti-CD3 binding fragment is attached to the C terminus of the light chain. Nevertheless an increased specific lysis of T-47D cells was induced by αMUC1-αCD3-CH3 compared to αMUC1-αCD3-Cκ. Similar to the TA-MUC1 and HER2 targeting constructs, using αTFα-αCD3-CH3 and pre-activated T cells as effector cells specific lysis of prostate cancer cell line DU-145 was induced (FIG. 6). As expected, none of the parental antibodies αMUC1 (FIG. 5A), αHER2 (FIG. 5B) and αTFα (FIG. 6), that do not recruit T cells, induced specific lysis when pre-activated T cells were used as effector cells.

For T cell ADCC assays with pre-activated T cells, T cells were first isolated by magnetic bead separation technic (Dynabeads® Untouched™ Human T cells Kit, Thermo Fisher) before the isolated T cells were activated for 5 days (Dynabeads® Human T-Activator CD3/CD28 for T cell Expansion and Activation, Thermo Fisher). This method of T cell pre-activation was chosen to get results that solely base on the process of binding to TA-MUC1 and release of perforin and granzymes leading to cell death, excluding potential differences in T cell activation that were analyzed in a different assay. Activated T cells were then used to compare the different bispecific constructs targeting TA-MUC1 or HER2. The assay was performed as a europium release assay. Briefly, target cells were loaded with europium (Eu³⁺) by electroporation and incubated with pre-activated T cells at an effector to target cell ratios (E:T ratio) of 10:1 in the presence of the bispecific constructs for 5 hours. Europium release to the supernatant (indicating antibody mediated cell death) was quantified using a fluorescence plate reader Infinite F200 (Tecan Austria GmbH). Maximal release was achieved by incubation of target cells with triton-X-100 and spontaneous release was measured in samples containing only target cells but no antibody and PBMCs.

Specific cytotoxicity was calculated as:

% specific lysis=(experimental release−spontaneous release)/(maximal release−spontaneous release)×100.

After T cell mediated ADCC was analyzed with pre-activated T cells, a different assay setup using unstimulated PBMCs as effector cells was established. Therefore, alongside TA-MUC1 or HER2 binding and release of perforin and granzymes, this assay setup additionally accounts for differences in T cell activation. Using unstimulated PBMCs as effector cells the results obtained with pre-activated T cells were confirmed. αMUC1-αCD3-Cκ and αMUC1-αCD3-CH3 (FIG. 5C) as well as αHER2-αCD3-Cκ and αHER2-αCD3-CH3 (FIG. 5D) showed effective lysis of TA-MUC1⁺ and HER2⁺ breast cancer cell line MCF-7. Again specific lysis mediated by αMUC1-αCD3-CH3 was enhanced compared with αMUC1-αCD3-Cκ (FIG. 5C) whereas αHER2-αCD3-Cκ showed stronger cytotoxic activity compared to αHER2-αCD3-CH3 (FIG. 5D). Again the results obtained with the HER2 binding constructs are in line with recent reports that strongly favor short distances between the T cell binding domain and the cancer cell binding domain. In contrast to this, an increased specific lysis of T-47D cells was induced by αMUC1-αCD3-CH3 compared to αMUC1-αCD3-Cκ. None of the parental antibodies αMUC1 (FIG. 5C) and αHER2 (FIG. 5D), that do not recruit T cells, induced specific lysis when unstimulated PBMCs were used as effector cells, indicating that at the low E:T ratio used in this experiments T cells are the main effector cell population that mediate cytotoxicity.

The PBMC ADCC assay was performed as a lactate dehydrogenase (LDH) release assay. Therefore TA-MUC1+ and HER2+ human breast cancer MCF-7 cells were seeded in 96-well flatbottom plates the day before the lysis assays. On the following day PBMCs were isolated from healthy donor buffy coats and added to the target cells at a final E:T ratio of 10:1 together with αMUC1, αMUC1-αCD3-Cκ, αMUC1-αCD3-CH3, αHER2, αHER2-αCD3-Cκ and αHER2-αCD3-CH3. After 24 h to 48 h target cell killing was assessed by quantification of lactate dehydrogenase (LDH) released into cell supernatant (Cytotoxicity Detection Kit (LDH), Roche). Maximal release was achieved by incubation of target cells with triton-X-100 and antibody independent cell death was measured in samples containing only target cells and PBMCs but no antibody.

### Example 6: Off-Target Effects

In order to analyze the safety profile of the bispecific antibody, the cytokine release of immune cells was determined depending on the presence of specific target cells of the antibody. Activation of immune cells and cytokine release in the absence of target cells are off-target effects which may lead to adverse side effects in human patients.

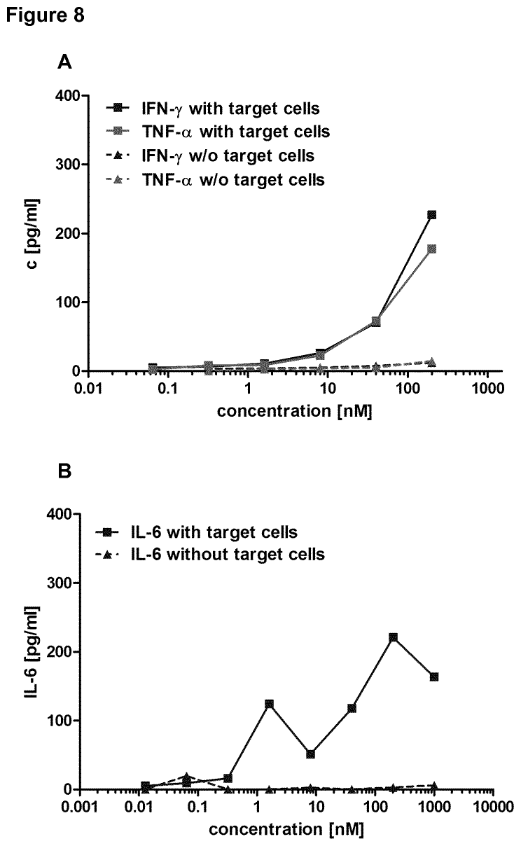

PBMCs were incubated with αMUC1-αCD3-CH3 with or without MUC1+ MCF-7 cells for 48h. After 48h, the supernatant was collected and the concentration of the cytokines IFN-γ, TNF-α and IL-6 was analyzed. In the presence of MUC1+ target cells, αMUC1-αCD3-CH3 mediated T cell activation leads to a dose-dependent cytokine release (see FIG. 8). In the absence of target cells, no cytokine release was detected. Hence, the bispecific antibody shows a very good target specificity with especially low off-target effects.

In another experiment, specificity of the bispecific antibody was analyzed using an untargeted T cell engaging bispecific antibody which does not bind to MUC1 but is otherwise identical to αMUC1-αCD3-CH3.

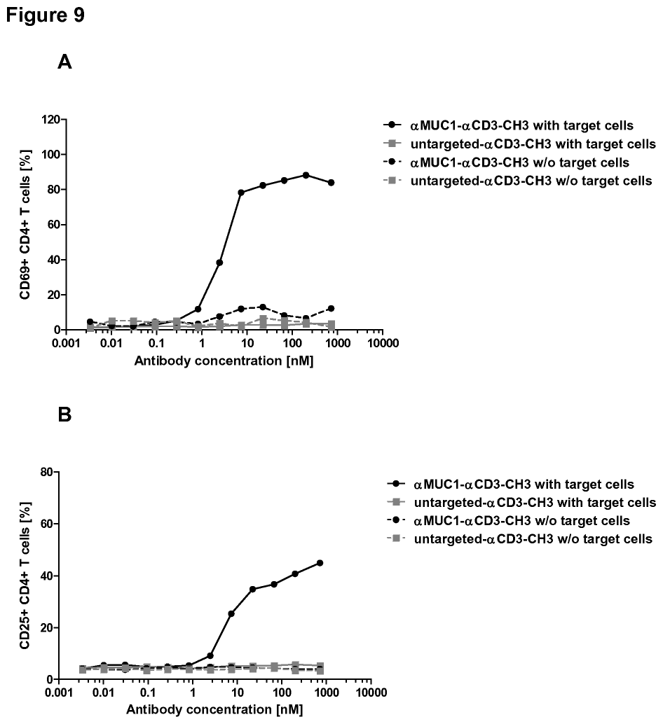

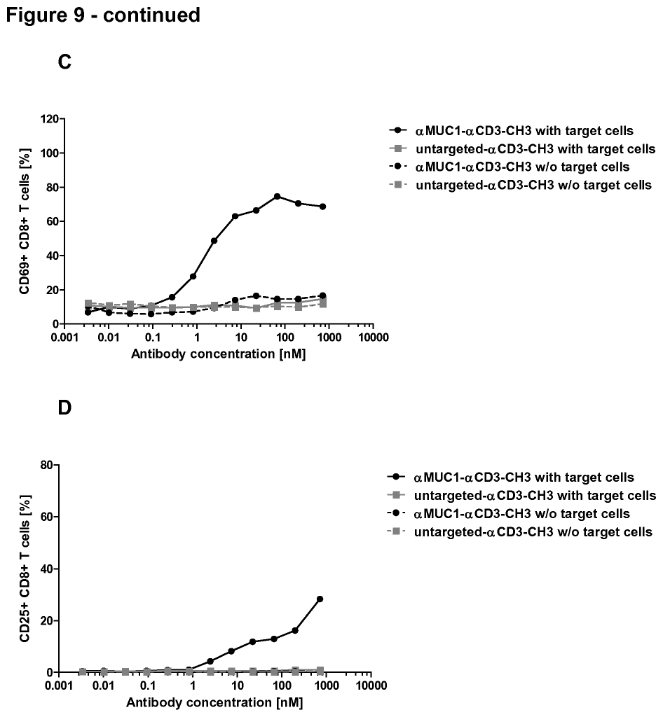

Binding to CD3+ Jurkat cells demonstrated that both bispecific antibodies have identical affinity to CD3 expressing immune cells (see FIG. 7). However, only the targeted bispecific antibody αMUC1-αCD3-CH3 is able to activate immune cells. Incubation of PBMCs with targeted or untargeted bispecific antibody and MUC1+ MCF-7 cells showed activation of CD4+ and CD8+ T cells only in the presence of targeted αMUC1-αCD3-CH3 (see FIG. 9). This demonstrates that only specific binding to the target cells and recruitment of the immune cells by the bispecific antibody αMUC1-αCD3-CH3 activates the immune cells.

### Example 7: T Cell Recruitment

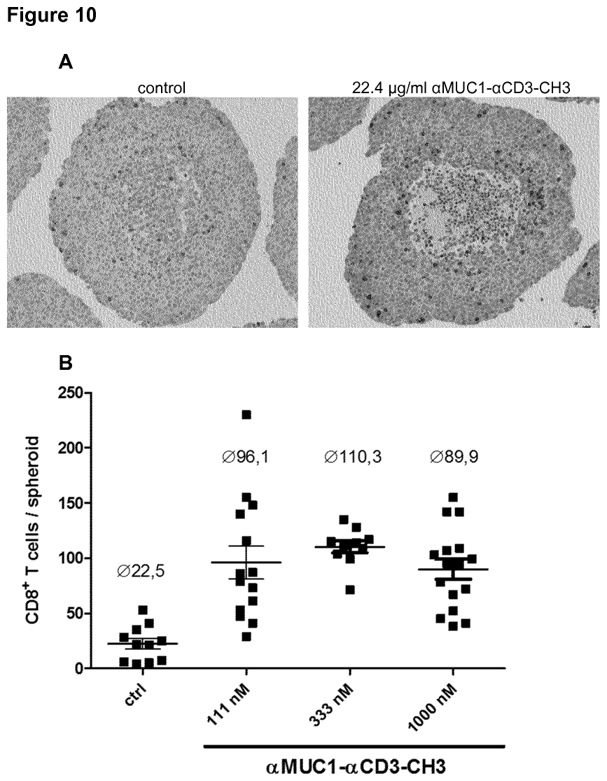

To demonstrate recruitment of immune cells to the tumor site, a 3D in vitro model was established. PBMCs were added to tumor spheroids and the coculture was stimulated with the bispecific antibody. After treatment with αMUC1-αCD3-CH3, the number of CD8+ T cells in the tumor spheroids markedly increased (see FIG. 10). Thus, the bispecific antibody effectively recruits immune cells to the tumor site.

### Example 8: Upregulation of MUC1

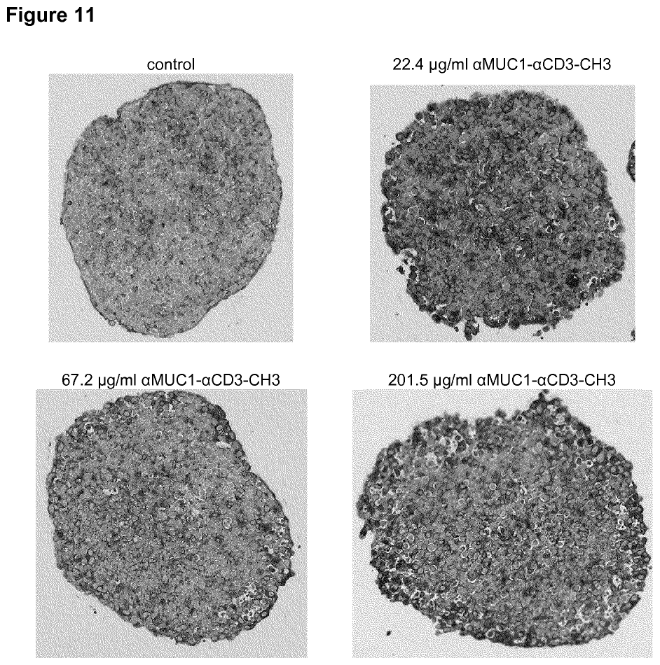

The effect of treatment with the bispecific antibody on the expression of tumor-associated MUC1 in the target tumor was analyzed using the 3D model of example 7. PBMCs were added to tumor spheroids and the coculture was stimulated with the bispecific antibody αMUC1-αCD3-CH3. After treatment with the antibody, upregulation of TA-MUC1 was observed (see FIG. 11). Therefore, after start of the therapy tumor targeting is even improved by the upregulated TA-MUC1 expression in the tumor cells.

### Example 9: Bystander Immune Cell Activation

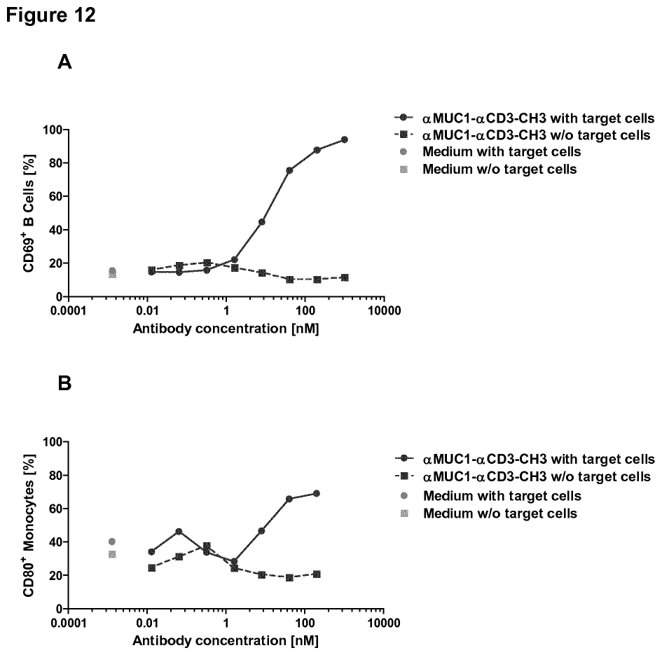

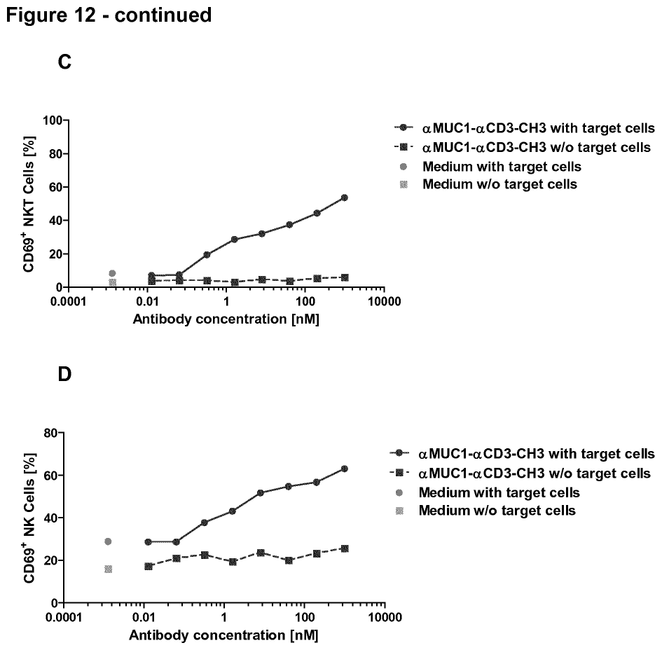

As a secondary effect, the activation of CD3+ immune cells, especially T cells, also results in the activation of further immune cells by the bispecific antibody. To demonstrate this, PBMCs were incubated with αMUC1-αCD3-CH3 in the presence of MUC1+ target cells for 48h. After 48h, different immune cell subsets were analyzed for expression of specific activation markers by flow cytometry (see FIG. 12). Incubation without target cells was used as negative control.

As shown in the graphs, αMUC1-αCD3-CH3 activated B cells, monocytes, NKT cells and NK cells in the presence of MUC1+ target cells. In the absence of these target cells, no activation of the immune cells was observed. These results demonstrate that αMUC1-αCD3-CH3 also activates bystander immune cells in the presence of MUC1+ target cells.

### Example 10: Analysis of Molecular Weight of the Intact Antibody Chains by LC-MS

Stability of the bispecific antibody was analyzed. αMUC1-αCD3-CH3 and αTFα-αCD3-CH3 were produced as described above in Example 1. In addition, a reference antibody of the same bispecific format but with mutated sequence between the C-terminus of the heavy chain and the linker sequence (the last amino acid of Fc part (K447) was deleted) was produced in a similar way. A stressed sample was produced by purification of the antibody from supernatant of a long batch production in a spinner flask.

Antibody samples were denatured and treated with DTT to reduce disulfide bridges. Subsequent deglycosylation is achieved by incubation with Rapid PNGase F (NEB). The sample was injected to a C4 column and analyzed by UPLC-MS employing a H₂O/AcN gradient with 0.1% formic acid. Mass spectra were acquired with a ESI-QTOF-MS (ImpactHD, Bruker). Resulting spectra were processed within Data Analysis (Bruker) employing background subtraction of 0.7 and MaxEnt algorithm for charge deconvolution between 20-80 kDa reflecting the expected mass range for the light chain (˜24 kDa) and the heavy chain (˜77 kDa).

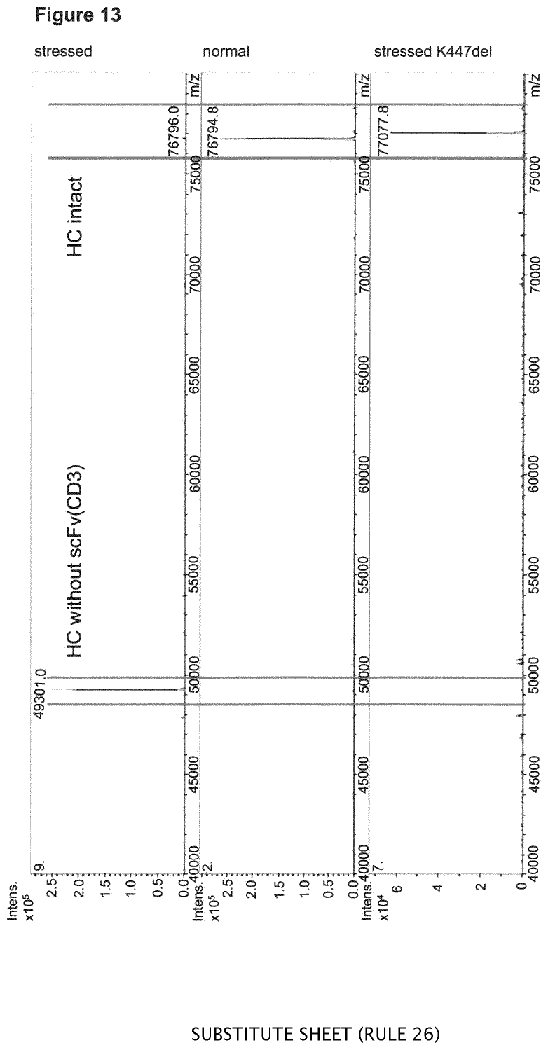

Signals for intact heavy chain of the bispecific antibodies (76.8 kDa) and a potential loss of the αCD3 single chain fragment at position K447 of the heavy chain resulting in a mass of 49.3 kDa were regarded. Results are summarized in FIG. 13. As demonstrated, the bispecific antibody produced as described in Example 1 (“normal”) does not show any degradation of the heavy chain. However, the stressed bispecific antibody derived from late stage of cultivation with high degree of dead cells shows degraded fragments (“stressed”). Sequence mutation as performed for the αTFα-αCD3-CH3 antibody (deletion of K447 in the heavy chain) stabilizes the construct because no fragments are detectable (“stressed K447del”).

### Identification of the Deposited Biological Material

The cell lines DSM ACC 2806, DSM ACC 2807 and DSM ACC 2856 were deposited at the DSMZ—Deutsche Sammlung von Mikroorganismen und Zellkulturen GmbH, Inhoffenstraße 7B, 38124 Braunschweig (DE) by Glycotope GmbH, Robert-Rössle-Str. 10, 13125 Berlin (DE) on the dates indicated in the following table.

| Name of the | Accession | | |
|---|---|---|---|
| Cell Line | Number | Depositor | Date of Deposition |
| NM-H9D8 | DSM ACC 2806 | Glycotope GmbH | Sep. 15, 2006 |
| NM-H9D8- | DSM ACC 2807 | Glycotope GmbH | Oct. 5, 2006 |
| E6 | | | |
| NM-H9D8- | DSM ACC 2856 | Glycotope GmbH | Aug. 8, 2007 |
| E6Q12 | | | |

1. A multispecific antibody construct, comprising
(i) an anti-MUC1 antibody module, the antibody module comprising at least one antibody heavy chain, and
(ii) an anti-CD3 antigen binding fragment;
wherein the antigen binding fragment is fused to the C terminus of the heavy chain of the antibody module.

2. The multispecific antibody construct according to claim 1, wherein the anti-MUC1 antibody module comprises
(a) two heavy chains each comprising a VH domain, a CH1 domain, a hinge region, a CH2 domain and a CH3 domain; and
(b) two light chains comprising a VL domain and a CL domain.

3. The multispecific antibody construct according to claim 1, wherein the anti-MUC1 antibody module specifically binds to a TA-MUC1 epitope and comprises an antibody heavy chain variable region, comprising
(i) an amino acid sequence which is at least 80% identical to any one of SEQ ID NOs: 7, 8 and 9, and
(ii) a set of heavy chain CDR sequences with CDR-H1 having the amino acid sequence of SEQ ID NO: 1, CDR-H2 having the amino acid sequence of SEQ ID NO: 3 and CDR-H3 having the amino acid sequence of SEQ ID NO: 5, or CDR-H1 having the amino acid sequence of SEQ ID NO: 2, CDR-H2 having the amino acid sequence of SEQ ID NO: 4 and CDR-H3 having the amino acid sequence of SEQ ID NO: 6.

4. The multispecific antibody construct according to claim 1, wherein the anti-MUC1 antibody module specifically binds to TFα and comprises an antibody heavy chain variable region, comprising
(i) an amino acid sequence which is at least 80% identical to any one of SEQ ID NOs: 27 to 32, and
(ii) a set of heavy chain CDR sequences with CDR-H1 having the amino acid sequence of SEQ ID NO: 21, CDR-H2 having the amino acid sequence of SEQ ID NO: 22 or 23 and CDR-H3 having the amino acid sequence of SEQ ID NO: 24, 25 or 26.

5. The multispecific antibody construct according to claim 1, wherein the anti-CD3 antigen binding fragment comprises a VH domain, a VL domain and a peptide linker between the VH domain and the VL domain; and in particular is an scFv fragment.

6. The multispecific antibody construct according to claim 1, wherein the anti-CD3 antigen binding fragment specifically binds to CD3ε and comprises an antibody heavy chain variable region, comprising
(i) an amino acid sequence which is at least 80% identical to any one of SEQ ID NOs: 46; and
(ii) a set of heavy chain CDR sequences with CDR-H1 having the amino acid sequence of SEQ ID NO: 43, CDR-H2 having the amino acid sequence of SEQ ID NO: 44 and CDR-H3 having the amino acid sequence of SEQ ID NO: 34.

7. The multispecific antibody construct according to claim 1, wherein the multispecific antibody construct comprises two anti-CD3 antigen binding fragments, each fused to the C terminus of a different heavy chain of the antibody module.

8. The multispecific antibody construct according to claim 1, wherein the antibody module does not comprise an N-glycosylation site in the CH2 domain.

9. The multispecific antibody construct according to claim 1, wherein the antibody module comprises an N-glycosylation site in the CH2 domain of the antibody heavy chains.

10. The multispecific antibody construct according to claim 9, wherein the antibody module has a glycosylation pattern in the CH2 domain of the antibody heavy chains, wherein the relative amount of glycans carrying a core fucose residue is at least 60% of the total amount of glycans attached to the CH2 domains of the antibody module in a composition of the multispecific antibody construct.

11. The multispecific antibody construct according to claim 9, wherein the antibody module has a glycosylation pattern in the CH2 domain of the antibody heavy chains, wherein the relative amount of glycans carrying a core fucose residue is 40% or less of the total amount of glycans attached to the CH2 domains of the antibody module in a composition of the multispecific antibody construct.

12. The multispecific antibody construct according to claim 1, comprising a further agent conjugated thereto.

13. A pharmaceutical composition comprising the multispecific antibody construct according to claim 1 and one or more further components selected from the group consisting of solvents, diluents, and excipients.

14. The multispecific antibody construct according to claim 1 for use in medicine.

15. A method for treating, prognosticating, diagnosing, and/or monitoring a diseases associated with abnormal cell growth in a patient, wherein the method comprises administering to the patient the multispecific antibody construct of claim 1, and wherein the disease is selected from the group consisting of cancer, infections such as bacterial, viral, fungal or parasitic infections, inflammatory diseases such as autoimmune diseases and inflammatory bowel diseases, graft-versus-host disease, and diseases associated with a reduce immune activity, such as immunodeficiencies.

16. A method of treating cancer in a patient, the method comprising administering to the patient the multispecific antibody construct of claim 1, wherein the cancer is selected from the group consisting of cancer of the breast, colon, stomach, liver, pancreas, kidney, blood, lung, and ovary.

17. A method for treating, prognosticating, diagnosing, and/or monitoring a disease associated with abnormal cell growth in a patient, wherein the method comprises administering to the patient the pharmaceutical composition of claim 13, and wherein the disease is selected from the group consisting of cancer, infections such as bacterial, viral, fungal or parasitic infections, inflammatory diseases such as autoimmune diseases and inflammatory bowel diseases, graft-versus-host disease, and diseases associated with a reduce immune activity, such as immunodeficiencies.

18. A method of treating cancer in a patient, the method comprising administering to the patient the pharmaceutical composition of claim 13, wherein the cancer is selected from the group consisting of cancer of the breast, colon, stomach, liver, pancreas, kidney, blood, lung, and ovary.
